SEQUENCE LISTING (31 sequences)

SEQ ID NO: 1 - DNA, 27 bp, Artificial
  An artificially synthesized primer for PCR
gtgaggtcagcctactactctctgaag

SEQ ID NO: 2 - DNA, 20 bp, Artificial
  An artificially synthesized primer for PCR
caggagggagtcaccgattg

SEQ ID NO: 3 - DNA, 23 bp, Artificial
  An artificially synthesized primer for PCR
aaggattatgaggaggttggtgt

SEQ ID NO: 4 - DNA, 23 bp, Artificial
  An artificially synthesized primer for PCR
cttgggtctgtaacaaagcattc

SEQ ID NO: 5 - DNA, 20 bp, Artificial
  An artificially synthesized primer for northern blot probe
atcaaaggccagatgattcg

SEQ ID NO: 6 - DNA, 20 bp, Artificial
  An artificially synthesized primer for northern blot probe
gacaactcccatgtggaacc

SEQ ID NO: 7 - DNA, 19 bp, Artificial
  An artificially synthesized target sequence for siRNA
gaagcaagtggagacactg

SEQ ID NO: 8 - DNA, 19 bp, Artificial
  An artificially synthesized target sequence for siRNA
gagccttggcaataagttc

SEQ ID NO: 9 - DNA, 19 bp, Artificial
  An artificially synthesized target sequence for siRNA
cattgtcaagcagaccatt

SEQ ID NO: 10 - DNA, 19 bp, Artificial
  An artificially synthesized target sequence for siRNA
gaagcagcacgacttcttc

SEQ ID NO: 11 - DNA, 34 bp, Artificial
  An artificially synthesized primer for PCR
agaattctgtgggcccctcatggcctcagcagga

SEQ ID NO: 12 - DNA, 35 bp, Artificial
  An artificially synthesized primer for PCR
tcgagcggccgcgatgggctcacagccaggaggga

SEQ ID NO: 13 - DNA, 30 bp, Artificial
  An artificially synthesized primer for PCR
gccctgggtcctggcagtcaacatttcccc

SEQ ID NO: 14 - DNA, 28 bp, Artificial
  An artificially synthesized primer for PCR
tgaattcatggcggcagccgttctgagt

SEQ ID NO: 15 - DNA, 34 bp, Artificial
  An artificially synthesized primer for PCR
tcgagcggccgcgaactgctgttgatattaataa

SEQ ID NO: 16 - DNA, 20 bp, Artificial
  An artificially synthesized primer for PCR
tttggcacagcaggagggga

SEQ ID NO: 17 - DNA, 29 bp, Artificial
  An artificially synthesized primer for PCR
tcgagcggccgcgaggagtcaggctctgt

SEQ ID NO: 18 - DNA, 4207 bp, Homo sapiens
gtcatgcgtggcggtgcgtggttgctaggggcgcctgaggctgccgggtagcccagcaggccgagggaggaagtagcgtggagccggtgccgagccggggcgaagctggatcccctagatagactgtcttcaagctcactgatattttcctctgcttgatccattgtgctgttgagagcctctagtaaatttttcagactgacagacttcaaggatgcagctgctactaccggaggtgtgtggcaccttacctcagcaaggccatgagaccgtgtggccatgatgtgggcccctcatggcctcagcaggaacacagcactatagtattggcctccgccagaaaagcagcttcaagcagagtggtccctcaggcacagtacctgccacgccacctgagaaaccctcggagggcagagtctggcctcaggcccatcagcaagtgaagccaatctggaagctggaaaagaagcaagtggagacactgtcagcagggttgggcccaggcctcttgggcgtcccaccccagccagcatatttcttttgccccagcactttatgtagctctgggaccacggctgtcattgcaggccacagcagttcctgttacctacactctctcccggacttgttcaacagcaccctgctataccgccgctccagctataggcaaaaaccgtaccagcaactggagtctttctgcttgcgttcgagcccgtcagaaaaaagccctttttctctccctcaaaagagcctccctgtcagtctcactgccaacaaggccacttcttccatggtcttctccatggcccagcccatggcctcctcatccacagaaccatacctctgcttggcagcggctggggaaaacccttcagggaagagcctggcctctgccatctcagggaagatcccatctccactctcttcctcctataagcccatgctgaataataattccttcatgtggccaaatagcacgccagtgcctttattgcagaccacacagggcctgaagccagtatcgccacccaagatccagcctgtctcctggcatcattcagggggtactggagactgtgcaccgcagcctgttgaccataaggtgcccaaaagcattggcactgtcccagctgatgccagtgcccatatcgccttgtctaccgctagctcccacgacacatccaccaccagtgttgcctcttcctggtataaccggaataacttagccatgagggcagagccactttcctgtgctctggatgacagctctgattcccaggatccaactaaggagattcggttcactgaggccgtgaggaaattgaccgcaagaggctttgagaagatgccgaggcaaggctgccagcttgaacagtctagtttcctgaaccccagcttccagtggaatgtcctcaacaggagcaggcggtggaaacctcctgcggtaaatcagcagtttcctcaggaggatgctggatcggtcaggcgggtcctccctggtgcctcagataccttggggttggacaatacagtcttctgtaccaagcgtatcagcattcacctccttgcctcacatgccagtgggctcaatcacaaccctgcctgtgaatctgtaattgactcctcagcatttggagaaggcaaagctccaggtcccccttttcctcaaactcttggcatagccaacgtggccacccgcctctcttccatccagctgggccagtctgagaaggagagacctgaggaggccagggagctggactcatctgatagggatattagttcagctactgacctccagccagatcaggctgagactgaagatacagaagaagaactagtagatagtttggaagactgttgtggccgtgatgagaatgaagaggaggagggagactcagagtgctcctcattaagtgctgtctcccccagcgaatcggtggccatgatctctagaagctgtatggaaattctgaccaaacccctttccaatcatgagaaagttgtccgaccagccctcatctacagtctctttcccaacgttccccctaccatctattttggcactcgggatgagagagtggagaaacttccctgggaacagaggaagttgctccgatggaagatgagcacagtgacccccaacattgtcaagcagaccattggacggtcccacttcaaaatcagcaaaagaaacgatgactggctgggctgctggggtcaccacatgaagtctcctagtttccgatccattcgagagcatcagaagctaaaccatttcccaggctcattccagattgggaggaaggaccggctatggcggaacctgtcacgtatgcagagccgctttggcaagaaggagttcagtttcttcccccagtcctttatcctgccccaggacgccaagctcctgcgcaaagcgtgggagagcagcagccgccaaaagtggattgtgaagccaccagcatcagctcgaggcattggcatccaggttattcacaagtggagtcagctccccaagcgaaggcccctcctggtacagaggtatctacacaaaccctacctcatcagcggcagcaagtttgacctgcggatctatgtttatgtcacttcctacgatcctctgcggatttacctcttttcagatggactggtccgctttgccagttgcaagtattcgccttccatgaagagccttggcaataagttcatgcacctgaccaactacagtgtcaataaaaagaatgccgagtaccaggccaatgcagatgaaatggcttgccagggccacaaatgggcactgaaggctttgtggaactacctgagccagaagggagtcaatagcgactccatctgggagaagataaaggatgttgttgtcaaaactatcatctcgtcagagccctatgtgaccagcctgctcaagatgtatgtgcgacggccctatagctgccatgaactctttggttttgacatcatgctagacgaaaacctcaagccctgggtcctggaagtcaacatttccccaagcctccactccagctctccactggatatcagcatcaaaggccagatgattcgtgaccttctgaatctggcaggttttgtcctgcccaatgcagaggatatcatttccagccccagcagctgcagcagctccaccaccagcctgcccacctcccctggggacaaatgtcgaatggctccagagcatgtcactgcacagaagatgaagaaagcctattatctgacccagaaaattcctgatcaggacttctatgcatctgtgctggatgtcctgacaccagatgatgttcggattctggttgagatggaagatgagttttctcgccgtggtcagtttgaacgaatttttccttctcatatctcctctcgctatctccgcttttttgagcagccacgatatttcaacattctcaccacccaatgggaacagaaataccatggcaacaagcttaaaggagtagatctgctccggagttggtgctacaaagggttccacatgggagttgtctctgattctgctccagtgtggtctctcccgacatcacttctgactatctcaaaggatgacgtgatactcaatgccttcagcaaatcagagactagcaagctgggaaaacaaagctcctgtgaggttagcctactactctctgaagacgggaccacgcccaaatccaagaagactcaagctggcctctccccttatccccagaaacccagttcctcaaaggacagtgaggacaccagcaaagagcccagcctttctacccagacgttacctgtgatcaagtgctctgggcagacttcaagactttctgcttcctccactttccagtcaatcagtgactccctcctggctgtgagcccataactggcctctctccaaaagcctctgcccaggagcatgggcatcagctacctcacgggaaccagcctgctgttcagaccagtctgaccccctacccctttcaccctgtccctcctcagagtattttttgaagtggttgcattatagagatgggtatttgtagggccggagggatggtagtgatggggagaaggtgaggaagggtcaccctctgtcacctgtctgcctggctggcacctcatatctcagcagagaagccagtggtggccacgcagccttataaagcaggttttggtttccaaaaaaaaaaaaaaa

SEQ ID NO: 19 - protein, 1199 aa, Homo sapiens
MASAGTQHYSIGLRQKSSFKQSGPSGTVPATPPEKPSEGRVWPQAHQQVKPIWKLEKKQVETLSAGLGPGLLGVPPQPAYFFCPSTLCSSGTTAVIAGHSSSCYLHSLPDLFNSTLLYRRSSYRQKPYQQLESFCLRSSPSEKSPFSLPQKSLPVSLTANKATSSMVFSMAQPMASSSTEPYLCLAAAGENPSGKSLASAISGKIPSPLSSSYKPMLNNNSFMWPNSTPVPLLQTTQGLKPVSPPKIQPVSWHHSGGTGDCAPQPVDHKVPKSIGTVPADASAHIALSTASSHDTSTTSVASSWYNRNNLAMRAEPLSCALDDSSDSQDPTKEIRFTEAVRKLTARGFEKMPRQGCQLEQSSFLNPSFQWNVLNRSRRWKPPAVNQQFPQEDAGSVRRVLPGASDTLGLDNTVFCTKRISIHLLASHASGLNHNPACESVIDSSAFGEGKAPGPPFPQTLGIANVATRLSSIQLGQSEKERPEEARELDSSDRDISSATDLQPDQAETEDTEEELVDSLEDCCGRDENEEEEGDSECSSLSAVSPSESVAMISRSCMEILTKPLSNHEKVVRPALIYSLFPNVPPTIYFGTRDERVEKLPWEQRKLLRWKMSTVTPNIVKQTIGRSHFKISKRNDDWLGCWGHHMKSPSFRSIREHQKLNHFPGSFQIGRKDRLWRNLSRMQSRFGKKEFSFFPQSFILPQDAKLLRKAWESSSRQKWIVKPPASARGIGIQVIHKWSQLPKRRPLLVQRYLHKPYLISGSKFDLRIYVYVTSYDPLRIYLFSDGLVRFASCKYSPSMKSLGNKFMHLTNYSVNKKNAEYQANADEMACQGHKWALKALWNYLSQKGVNSDSIWEKIKDVVVKTIISSEPYVTSLLKMYVRRPYSCHELFGFDIMLDENLKPWVLEVNISPSLHSSSPLDISIKGQMIRDLLNLAGFVLPNAEDIISSPSSCSSSTTSLPTSPGDKCRMAPEHVTAQKMKKAYYLTQKIPDQDFYASVLDVLTPDDVRILVEMEDEFSRRGQFERIFPSHISSRYLRFFEQPRYFNILTTQWEQKYHGNKLKGVDLLRSWCYKGFHMGVVSDSAPVWSLPTSLLTISKDDVILNAFSKSETSKLGKQSSCEVSLLLSEDGTTPKSKKTQAGLSPYPQKPSSSKDSEDTSKEPSLSTQTLPVIKCSGQTSRLSASSTFQSISDSLLAVSP

SEQ ID NO: 20 - DNA, 3688 bp, Homo sapiens
acaggcagcctcatccgccatcactccgggaaccgagcaccccgctgcctcggcgcccaggctgctctctccgcgcatgctccttcccagcttgagcccgcgagtccagcctgccttcactcagtgcgcaagaccgcagccccctccccacgccccctccccagtaggcggccgtcgcggtgtgtttgcgtcatcgcgccacgccaccactggaacccgggggaagatggcggcagccgttctgagtgggccctctgcgggctccgcggctggggttcctggcgggaccgggggtctctcggcagtgagctcgggcccgcggctccgcctgctgctgctggagagtgtttctggtttgctgcaacctcgaacggggtctgccgttgctccggtgcatcccccaaaccgctcggccccacatttgcccgggctcatgtgcctattgcggctgcatgggtcggtgggcggggcccagaacctttcagctcttggggcattggtgagtctcagtaatgcacgtctcagttccatcaaaactcggtttgagggcctgtgtctgctgtccctgctggtaggggagagccccacagagctattccagcagcactgtgtgtcttggcttcggagcattcagcaggtgttacagacccaggacccgcctgccacaatggagctggccgtggctgtcctgagggacctcctccgatatgcagcccagctgcctgcactgttccgggacatctccatgaaccacctccctggccttctcacctccctgctgggcctcaggccagagtgtgagcagtcagcattggaaggaatgaaggcttgtatgacctatttccctcgggcttgtggttctctcaaaggcaagctggcctcattttttctgtctagggtggatgccttgagccctcagctccaacagttggcctgtgagtgttattcccggctgccctctttaggggctggcttttcccaaggcctgaagcacaccgagagctgggagcaggagctacacagtctgctggcctcactgcacaccctgctgggggccctgtacgagggagcagagactgctcctgtgcagaatgaaggccctggggtggagatgctgctgtcctcagaagatggtgatgcccatgtccttctccagcttcggcagaggttttcgggactggcccgctgcctagggctcatgctcagctctgagtttggagctcccgtgtccgtccctgtgcaggaaatcctggatttcatctgccggaccctcagcgtcagtagcaagaatattagcttgcatggagatggtcccctgcggctgctgctgctgccctctatccaccttgaggccttggacctgctgtctgcactcatcctcgcgtgtggaagccggctcttgcgctttgggatcctgatcggccgcctgcttccccaggtcctcaattcctggagcatcggtagagattccctctctccaggccaggagaggccttacagcacggttcggaccaaggtgtatgcgatattagagctgtgggtgcaggtttgtggggcctcggcgggaatgcttcagggaggagcctctggagaggccctgctcacccacctgctcagcgacatctccccgccagctgatgcccttaagctgcgtagcccgcgggggagccctgatgggagtttgcagactgggaagcctagcgcccccaagaagctaaagctggatgtgggggaagctatggccccgccaagccaccggaaaggggatagcaatgccaacagcgacgtgtgtgcggctgcactcagaggcctcagccggaccatcctcatgtgtgggcctctcatcaaggaggagactcacaggagactgcatgacctggtcctccccctggtcatgggtgtacagcagggtgaggtcctaggcagctccccgtacacgagctcccgctgccgccgtgaactctactgcctgctgctggcgctgctgctggccccgtctcctcgctgcccacctcctcttgcctgtgccctgcaagccttctccctcggccagcgagaagatagccttgaggtctcctctttctgctcagaagcactggtgacctgtgctgctctgacccacccccgggttcctcccctgcagcccatgggccccacctgccccacacctgctccagttccccctcctgaggccccatcgcccttcagggccccaccgttccatcctccgggccccatgccctcagtgggctccatgccctcagcaggccccatgccttcagcaggccccatgccctcagcaggccctgtgccctcggcacgccctggacctcccaccacagccaaccacctaggcctttctgtcccaggcctagtgtctgtccctccccggcttcttcctggccctgagaaccaccgggcaggctcaaatgaggaccccatccttgcccctagtgggactcccccacctactatacccccagatgaaacttttggggggagagtgcccagaccagcctttgtccactatgacaaggaggaggcatctgatgtggagatctccttggaaagtgactctgatgacagcgtggtgatcgtgcccgaggggcttccccccctgccacccccaccaccctcaggtgccacaccaccccctatagcccccactgggccaccaacagcctcccctcctgtgccagcgaaggaggagcctgaagaacttcctgcagccccagggcctctcccgccacccccacctccgccgccgcctgttcctggtcctgtgacgctccctccaccccagttggtccctgaagggactcctggtgggggaggacccccagccctggaagaggatttgacagttattaatatcaacagcagtgatgaagaggaggaggaagaggaagaagaggaagaagaagaagaggaagaagaggaagaggaggaagactttgaggaagaggaagaggatgaagaggaatattttgaagaggaagaagaggaggaagaagagtttgaggaagaatttgaggaagaagaaggtgagttagaggaagaagaagaagaggaggatgaggaggaggaagaagaactggaagaggtggaagacctggagtttggcacagcaggaggggaggtagaagaaggtgcacctccacccccaaccctgcctccagctctgcctccccctgagtctcccccaaaggtgcagccagaacccgaacctgaacccgggctgcttttggaagtggaggagccagggacggaggaggagcgtggggctgacacagctcccaccctggcccctgaagcgctcccctcccagggagaggtggagagggaaggggaaagccctgcggcagggccccctccccaggagcttgttgaagaagagccctctgctcccccaaccctgttggaagaggagactgaggatgggagtgacaaggtgcagcccccaccagagacacctgcagaagaagagatggagacagagacagaggccgaagctctccaggaaaaggagcaggatgacacagctgccatgctggccgacttcatcgattgtccccctgatgatgagaagccaccacctcccacagagcctgactcctagccatcttctgcaccccactctttgtttccaataaagttatgtccttagatagcgaaaaaaaaaaaaaaa

SEQ ID NO: 21 - protein, 1130 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPPADALKLRSPRGSPDGSLQTGKPSAPKKLKLDVGEAMAPPSHRKGDSNANSDVCAAALRGLSRTILMCGPLIKEETHRRLHDLVLPLVMGVQQGEVLGSSPYTSSRCRRELYCLLLALLLAPSPRCPPPLACALQAFSLGQREDSLEVSSFCSEALVTCAALTHPRVPPLQPMGPTCPTPAPVPPPEAPSPFRAPPFHPPGPMPSVGSMPSAGPMPSAGPMPSAGPVPSARPGPPTTANHLGLSVPGLVSVPPRLLPGPENHRAGSNEDPILAPSGTPPPTIPPDETFGGRVPRPAFVHYDKEEASDVEISLESDSDDSVVIVPEGLPPLPPPPPSGATPPPIAPTGPPTASPPVPAKEEPEELPAAPGPLPPPPPPPPPVPGPVTLPPPQLVPEGTPGGGGPPALEEDLTVININSSDEEEEEEEEEEEEEEEEEEEEEDFEEEEEDEEEYFEEEEEEEEEFEEEFEEEEGELEEEEEEEDEEEEEELEEVEDLEFGTAGGEVEEGAPPPPTLPPALPPPESPPKVQPEPEPEPGLLLEVEEPGTEEERGADTAPTLAPEALPSQGEVEREGESPAAGPPPQELVEEEPSAPPTLLEEETEDGSDKVQPPPETPAEEEMETETEAEALQEKEQDDTAAMLADFIDCPPDDEKPPPPTEPDS

SEQ ID NO: 22 - protein, 293 aa, Artificial
  TTL domain
REHQKLNHFPGSFQIGRKDRLWRNLSRMQSRFGKKEFSFFPQSFILPQDAKLLRKAWESSSRQKWIVKPPASARGIGIQVIHKWSQLPKRRPLLVQRYLHKPYLISGSKFDLRIYVYVTSYDPLRIYLFSDGLVRFASCKYSPSMKSLGNKFMHLTNYSVNKKNAEYQANADEMACQGHKWALKALWNYLSQKGVNSDSIWEKIKDVVVKTIISSEPYVTSLLKMYVRRPYSCHELFGFDIMLDENLKPWVLEVNISPSLHSSSPLDISIKGQMIRDLLNLAGFVLPNAEDII

SEQ ID NO: 23 - protein, 78 aa, Artificial
  glutamate rich region
DEEEEEEEEEEEEEEEEEEEEEDFEEEEEDEEEYFEEEEEEEEEFEEEFEEEEGELEEEEEEEDEEEEEELEEVEDLE

SEQ ID NO: 24 - DNA, 32 bp, Artificial
  mutant PELP1 primer sequence
tcgagcggccgcaactccaggtcttccacctc

SEQ ID NO: 25 - DNA, 30 bp, Artificial
  SENP3 primer sequence
attcgcggccgcatgaaagagactatacaa

SEQ ID NO: 26 - DNA, 28 bp, Artificial
  SENP3 primer sequence
ccgctcgagcacagtgagtttgcagtga

SEQ ID NO: 27 - DNA, 23 bp, Artificial
  LAS1L primer sequence
tgaattcatgtcgtgggaatccg

SEQ ID NO: 28 - DNA, 2495 bp, Homo sapiens
ggagtgagggtagcacgctgagctgaaggctgtgcggagcggcgcggcacagagcctgttgttgagctcagtatgtcgtgggaatccggggccgggccaggtctaggttcccaggggatggatctcgtgtggagtgcgtggtacggaaagtgcgttaaagggaaagggtcgttgccactctcggcccacggcatcgtggtcgcctggctcagcagggccgagtgggaccaggtgacggtttatctgttctgtgacgaccataagttgcagcggtacgcgcttaaccgcatcacggtgtggaggagcaggtcaggcaacgaactccctctggcagtggcttctactgctgacctgatacgctgtaagctcttggatgtaactggtggcttgggcactgatgaacttagactgctctatggcatggcattggtcaggtttgtgaatcttatctcagagaggaagacaaagtttgccaaggtccccctcaagtgtctggctcaagaggtaaatattccggattggattgttgaccttcgccatgagttgacccacaagaaaatgccccatataaatgactgccgcagaggctgctactttgtcctggattggctccagaagacctattggtgccgccaactggagaacagcctgagagagacctgggagttggaggagttcagggaagggatagaggaagaggatcaagaggaagataagaacattgttgttgatgacatcacagaacagaaaccagagcctcaggatgatgggaaaagtacggagtcagatgtaaaggccgatggagacagcaaaggcagcgaagaggtggattctcattgcaaaaaggccctgagtcataaagagctatatgaaagagcccgagaactgctggtatcatacgaagaggagcagtttacggtgctggagaaatttaggtatttacctaaggccattaaggcgtggaataacccgtccccacgtgtagaatgtgtcctggcagagctcaagggcgttacatgcgagaacagggaggctgtgctggatgcttttctggatgatggcttccttgtccccacatttgaacagttggcagctttgcagatagaatatgaagatggtcagactgaggtccagagaggggaaggtactgacccaaagtcacacaaaaacgtggacttgaatgacgtcctggtgccaaagccgttctctcagttctggcagcccctgctcaggggcctgcactcccagaacttcacgcaggccctattggagaggatgctctctgaactgccagccttggggatcagcgggatccggcctacctacatcctcagatggaccgttgaactgatcgtggccaacaccaagactggacggaatgctcgccgattttctgcaggccagtgggaagcaagaaggggctggaggctgttcaactgctccgcctcccttgactggccccggatggttgagtcctgcttgggctcaccttgctgggccagcccccaactccttcggatcatcttcaaagccatggggcagggcctgccagacgaggagcaggagaagctgctgcgcatctgttccatttatacccagagtggagaaaacagcctggtgcaggagggctctgaggcctcccccattgggaagtctccatatacactagacagcctgtattggagcgtcaagccagccagctccagcttcgggtctgaagcaaaggcccagcaacaggaggagcagggcagtgttaatgatgtcaaggaagaggagaaggaggagaaagaggtcttgccagaccaggtagaggaggaggaagaaaatgatgaccaagaggaggaagaggaggatgaagatgatgaagatgatgaagaggaagacagaatggaggtggggcctttctctacagggcaagagtcccccactgccgagaatgctaggcttctggcccagaaaagaggagctttgcagggctctgcatggcaggttagctcagaagacgtgcgatgggacacatttcccctaggccgaatgccaggtcagaccgaggacccagcagagctcatgctggagaattatgacaccatgtatcttttggaccagcctgtgctagagcagcggctggaaccctcaacatgcaagactgacaccttgggcctgagctgtggtgtcggcagtggcaactgcagcaacagcagcagcagcaacttcgagggccttctctggagccaggggcagctgcatgggctcaaaactggcctgcagctcttctgatggccatccctggtgcaagtgttcatccagccgtgccagggcaacagcccaccccctagtacaactgatgctccctgagacaacctgggagacagcctggatcagccacatcaactcagttgtccaccacaggggaattttgaatgtcttttgtttttgttttgttttgaaaaataataaacaggcacccaaaaaaaaaaaaaaaaaaaaaaaaaaaa

SEQ ID NO: 29 - protein, 734 aa, Homo sapiens
MSWESGAGPGLGSQGMDLVWSAWYGKCVKGKGSLPLSAHGIVVAWLSRAEWDQVTVYLFCDDHKLQRYALNRITVWRSRSGNELPLAVASTADLIRCKLLDVTGGLGTDELRLLYGMALVRFVNLISERKTKFAKVPLKCLAQEVNIPDWIVDLRHELTHKKMPHINDCRRGCYFVLDWLQKTYWCRQLENSLRETWELEEFREGIEEEDQEEDKNIVVDDITEQKPEPQDDGKSTESDVKADGDSKGSEEVDSHCKKALSHKELYERARELLVSYEEEQFTVLEKFRYLPKAIKAWNNPSPRVECVLAELKGVTCENREAVLDAFLDDGFLVPTFEQLAALQIEYEDGQTEVQRGEGTDPKSHKNVDLNDVLVPKPFSQFWQPLLRGLHSQNFTQALLERMLSELPALGISGIRPTYILRWTVELIVANTKTGRNARRFSAGQWEARRGWRLFNCSASLDWPRMVESCLGSPCWASPQLLRIIFKAMGQGLPDEEQEKLLRICSIYTQSGENSLVQEGSEASPIGKSPYTLDSLYWSVKPASSSFGSEAKAQQQEEQGSVNDVKEEEKEEKEVLPDQVEEEEENDDQEEEEEDEDDEDDEEEDRMEVGPFSTGQESPTAENARLLAQKRGALQGSAWQVSSEDVRWDTFPLGRMPGQTEDPAELMLENYDTMYLLDQPVLEQRLEPSTCKTDTLGLSCGVGSGNCSNSSSSNFEGLLWSQGQLHGLKTGLQLF

SEQ ID NO: 30 - DNA, 2499 bp, Homo sapiens
ggcggcggtggcgctggtggcggcggtggcggaggtggaggtggaggtggaagctgaagctgaagcagagccagaggcggcgcggcggggtgctcggcggcgcggcacgcggcccagaagcgttggaatcctgaattgagacggctgcgcctaaagacagcaggagtggcggggccgccgccgtcgccggagagatgagccgggaagcttgaggccggagacgcccgccttcgggcccgtccgcccggcttccccgctcccgggtactggaagatgaaagagactatacaagggaccgggtcctgggggcctgagcctcctggacccggcatacccccagcttactcaagtcccaggcgggagcgtcttcgttggcccccacctcccaaaccccgactcaagtcaggtggagggtttgggccagatcctgggtcagggaccacagtgccagccagacgcctccctgtcccccgaccctcttttgatgcctcagcaagtgaagaggaggaagaagaggaggaggaggaggatgaagatgaagaggaggaagtggcagcttggaggctgcccccaagatggagtcagctgggaacctcccagcggccccgcccttcccgccccactcatcgaaaaacctgctcacagcgccgccgccgagccatgagagccttccggatgctgctctactcaaaaagcacctcgctgacattccactggaagctttgggggcgccaccggggccggcggcggggcctcgcacaccccaagaaccatctttcaccccagcaagggggtgcgacgccacaggtgccatccccctgttgtcgttttgactccccccgggggccacctccaccccggctgggtctgctaggtgctctcatggctgaggatggggtgagagggtctccaccagtgccctctgggccccccatggaggaagatggactcaggtggactccaaagtctcctctggaccctgactcgggcctcctttcatgtactctgcccaacggttttgggggacaatctgggccagaaggggagcgcagcttggcaccccctgatgccagcatcctcatcagcaatgtgtgcagcatcggggaccatgtggcccaggagctttttcagggctcagatttgggcatggcagaagaggcagagaggcctggggagaaagccggccagcacagccccctgcgagaggagcatgtgacctgcgtacagagcatcttggacgaattccttcaaacgtatggcagcctcatacccctcagcactgatgaggtagtagagaagctggaggacattttccagcaggagttttccaccccttccaggaagggcctggtgttgcagctgatccagtcttaccagcggatgccaggcaatgccatggtgaggggcttccgagtggcttataagcggcacgtgctgaccatggatgacttggggaccttgtatggacagaactggctcaatgaccaggtgatgaacatgtatggagacctggtcatggacacagtccctgaaaaggtgcatttcttcaatagtttcttctatgataaactccgtaccaagggttatgatggggtgaaaaggtggaccaaaaacgtggacatcttcaataaggagctactgctaatccccatccacctggaggtgcattggtccctcatctctgttgatgtgaggcgacgcaccatcacctattttgactcgcagcgtaccctaaaccgccgctgccctaagcatattgccaagtatctacaggcagaggcggtaaagaaagaccgactggatttccaccagggctggaaaggttacttcaaaatgaatgtggccaggcagaataatgacagtgactgtggtgcttttgtgttgcagtactgcaagcatctggccctgtctcagccattcagcttcacccagcaggacatgcccaaacttcgtcggcagatctacaaggagctgtgtcactgcaaactcactgtgtgagcctcgtaccccagaccccaagcccataaatgggaagggagacatgggagtcccttcccaagaaactccagttcctttcctctcttgcctcttcccactcacttccctttggtttttcatatttaaatgtttcaatttctgtatttttttttctttgagagaatacttgttgatttctgatgtgcagggggtggctacagaaaagcccctttcttcctctgtttgcaggggagtgtggccctgtggcctgggtggagcagtcatcctcccccttccccgtgcagggagcaggaaatcagtgctgggggtggtgggcggacaataggatcactgcctgccagatcttcaaacttttatatatatatatatatatatatatatatatatatatatatatatatatatatatatatatatatatatataaaaatatataaatgccacggtcctgctctggtcaataaaggatcctttgttgatacgtaaaaaaaaaaaaaaaaa

SEQ ID NO: 31 - protein, 574 aa, Homo sapiens
MKETIQGTGSWGPEPPGPGIPPAYSSPRRERLRWPPPPKPRLKSGGGFGPDPGSGTTVPARRLPVPRPSFDASASEEEEEEEEEEDEDEEEEVAAWRLPPRWSQLGTSQRPRPSRPTHRKTCSQRRRRAMRAFRMLLYSKSTSLTFHWKLWGRHRGRRRGLAHPKNHLSPQQGGATPQVPSPCCRFDSPRGPPPPRLGLLGALMAEDGVRGSPPVPSGPPMEEDGLRWTPKSPLDPDSGLLSCTLPNGFGGQSGPEGERSLAPPDASILISNVCSIGDHVAQELFQGSDLGMAEEAERPGEKAGQHSPLREEHVTCVQSILDEFLQTYGSLIPLSTDEVVEKLEDIFQQEFSTPSRKGLVLQLIQSYQRMPGNAMVRGFRVAYKRHVLTMDDLGTLYGQNWLNDQVMNMYGDLVMDTVPEKVHFFNSFFYDKLRTKGYDGVKRWTKNVDIFNKELLLIPIHLEVHWSLISVDVRRRTITYFDSQRTLNRRCPKHIAKYLQAEAVKKDRLDFHQGWKGYFKMNVARQNNDSDCGAFVLQYCKHLALSQPFSFTQQDMPKLRRQIYKELCHCKLTV

(19) United States
(12) Patent Application Publication
(10) Pub. No.: US 2011/0251090 A1
(43) Pub. Date: Oct. 13, 2011

(54) PANCREATIC CANCER RELATED GENE TTLL4

(75) Inventors: Yusuke Nakamura, Tokyo (JP); Hidewaki Nakagawa, Tokyo (JP); Akira Togashi, Kanagawa (JP)

(73) Assignee: Oncotherapy Science, Inc., Kanagawa (JP)

(21) Appl. No.: 13/061,071

(22) PCT Filed: Aug. 21, 2009
(86) PCT No.: PCT/JP2009/004031

§ 371 Date: Jun. 24, 2011

Related U.S. Application Data
(60) Provisional application No. 61/190,396, filed on Aug. 27, 2008.

(51) Int. Cl. C40B 30/04; C07K 16/40; C12Q 1/68; C07H 21/00; C12Q 1/25; G01N 33/53

(52) U.S. Cl. 506/9; 435/4; 435/7.8; 435/6.13; 435/29; 536/24.31; 530/389.1; 435/7.1

(57) ABSTRACT

The present invention relates to the roles played by TTLL4 genes in pancreatic cancer carcinogenesis and features a method for treating or preventing pancreatic cancer by administering a double-stranded molecule against one or more of the TTLL4 genes or a composition, vector or cell containing such a double stranded molecule. Also, disclosed are methods of identifying compounds for treating and preventing pancreatic cancer, using as an index their effect on the over-expression of TTLL4 in the pancreatic cancer cell.

### TECHNICAL FIELD

The present application claims the benefit of U.S. Provisional Application No. 61/190,396, filed on Aug. 27, 2008, the entire contents of which are incorporated by reference herein.

The present invention relates to methods of detecting and diagnosing pancreatic cancer as well as methods of treating and preventing pancreatic cancer. In particular, the present invention relates to TTLL4.

### BACKGROUND ART

Pancreatic ductal adenocarcinoma (PDAC) is the fourth leading cause of cancer death in the western world and has the worst mortality among common malignancies, with a 5-year survival rate of only 5% (NPLs 1-2). In 2007, it is estimated that 37,170 new cases of pancreatic cancer are diagnosed and a roughly equal number of deaths are attributed to pancreatic cancer in the United States (NPL 3). The majority of PDAC patients are diagnosed at an advanced stage, for which no effective therapy is available at present. Although only surgical resection offers a little possibility for cure, 80-90% of PDAC patients who undergo curative surgery die from their disseminated or metastatic diseases (NPLs 1-2). Recent advances in surgery and chemotherapy including 5-FU or gemcitabine, with or without radiation, can improve patients' quality of life (NPLs 1-2), but those treatments have a very limited effect on long-term survival of PDAC patients due to their extremely aggressive and chemo-resistant nature. Hence, the management of most patients is focused on palliative measures (NPL 1). To overcome this dismal situation, development of novel molecular therapies against good molecular targets is an urgent issue. Toward this direction, the present inventors previously generated detailed gene-expression profiles of PDAC cells using genome-wide cDNA microarrays consisting of approximately 30,000 genes, in combination with laser microbeam microdissection to enrich populations of cancer cells (NPL 4).

TTLL4 (tubulin tyrosine ligase-like family member 4) is a member of a large family of proteins with a TTL homology domain, whose members could catalyze ligations of diverse amino acids to tubulins or other substrate, such as tyrosination, polyglycylation, and polyglutamylation (NPL 5). Recently, it has been proved that some TTLL family members have activity to polyglutamylate tubulins and microtubule (MT)-associated proteins (NPL 6). Polyglutamylation is a post-translational modification in which glutamate side chains of variable lengths are formed on the target protein, which was first discovered on tubulins, and it is likely to influence their stability and the interaction between MTs and their associated proteins (NPLs 5-6). Furthermore, it was demonstrated that TTLL4 and TTLL5 could polyglutamylate several non-tubulin proteins as well as tubulins (NPL 7).

### CITATION LIST

### Non Patent Literature

NPL 1: DiMagno E P et al., 1999 Gastroenterology 117:1464-84
NPL 2: Zervos E E et al., 2004 Cancer Control 11: 23-31
NPL 3: Jemal A et al., 2007 CA Cancer J Clin 57: 43-66
NPL 4: Nakamura T et al., Oncogene 2004, 23: 2385-400
NPL 5: Westermann S et al., Nature Rev Mol Cell Biol 2003, 4: 938-947
NPL 6: Janke C et al., Science 2005, 308: 1758-1762
NPL 7: van Dijk J, et al., J Bio Chem 2008, 283: 3915-3922

### SUMMARY OF INVENTION

In this invention, over-expression of TTLL4 was identified in cancer cells and its role in cancer viability. The inventors have also demonstrated that TTLL4 polyglutamylates a signal-scaffold protein, PELP1 (prolin-glutamic acid-leucine-rich protein 1) in its glutamate-rich stretch region, and this polyglutamylation modifies the function of PELP1 in cancer cells. As such, the present invention relates to novel compositions and methods for detecting, diagnosing, treating and/or preventing cancer as well as methods for screening for useful agents.

In particular, the present invention arises from the discovery that double-stranded molecules composed of specific sequences (in particular, SEQ ID NOs: 7 and 8) are effective for inhibiting cellular growth of cancer cells, in particular pancreatic cancer cells. Specifically, small interfering RNAs (siRNAs) targeting TTLL4 genes are provided by the present invention. These double-stranded molecules may be utilized in an isolated state or encoded in vectors and expressed from the vectors. Accordingly, it is an object of the present invention to provide such double stranded molecules as well as vectors and host cells expressing them.

In one aspect, the present invention provides methods for inhibiting cancer cell growth and treating cancer by administering the double-stranded molecules or vectors of the present invention to a subject in need thereof. Such methods encompass administering to a subject a composition composed of one or more of the double-stranded molecules or vectors.

In another aspect, the present invention provides compositions for treating a cancer containing at least one of the double-stranded molecules or vectors of the present invention.

In yet another aspect, the present invention provides a method of diagnosing or determining a predisposition to pancreatic cancer in a subject by determining an expression level of TTLL4 in a patient derived biological sample. An increase in the expression level of one or more of the genes as compared to a normal control level of the genes indicates that the subject suffers from or is at risk of developing pancreatic cancer.

In a further aspect, the present invention provides a method of screening for a compound for treating and/or preventing pancreatic cancer. Such a compound would bind with TTLL4 gene or reduce the biological activity of TTLL4 gene or reduce the expression of TTLL4 gene or reporter gene used as a surrogate for the TTLL4 gene. Moreover, compounds that inhibit the binding between PELP1 and TTLL4 are useful to reduce a symptom of cancer.

In yet further aspect, the present invention further provides methods of identifying an agent that inhibits the polyglutamylation of PELP1 by TTLL4, by contacting a test cell expressing TTLL4 and PELP1 with a test compound and selecting the test compound reducing the polyglutamylated level of PELP1.

The present invention further provides methods of identifying a candidate agent for treating and/or preventing pancreatic cancer by contacting a polypeptide of the invention with a polygutamylated substrate and a glutamate under conditions suitable for polyglutamylation of the substrate in the presence of a test agent and selecting the test compound reducing the polyglutamylated level of the substrate.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF DRAWINGS

FIG. 1 shows the TTLL4 over-expression in PDAC cells. (A) Semi-quantitative RT-PCR validated that TTLL4 was over-expressed in the microdissected PDAC cells, compared with normal pancreatic ductal cells which were also microdissected (NPD), total normal pancreatic tissue (Panc), and vital organs (heart, lung, liver, and kidney). Expression of TUBA served as the quantitative control. (B) Multiple Tissue Northern blot analysis showed TTLL4 expression only in the testis, among the human adult organs. (C) Northern blot analysis for TTLL4 expression showed that all of the eight examined PDAC cell lines (Panc-1, Mia-PaCa-2, KLM-1, SUIT-2, KP-1N, PK-1, PK-45P, and PK-59) expressed TTLL4, while the normal adult organs did not express TTLL4, except for the testis.

FIG. 2 shows the effect of TTLL4-siRNA on growth of PDAC cells. (A) RT-PCR confirmed knockdown effect on TTLL4 expression by si#466 and si#2692, but not by si#2146 and a negative control siEGFP in Panc-1 (left) and Mia-PaCa-2 (right) cells. Beta 2MG was used to quantify RNAs. (B) Colony formation assay of Panc-1(left) and Mia-PaCa-2 (right) cells transfected with each of indicated siRNA-ex-pressing vectors to TTLL4 (si#466 and si#2692, and si#2146) and a negative control vector (siEGFP). Cells were visualized with 0.1% crystal violet staining after 2 weeks incubation with Geneticin. (C) MTT assay of each of Panc-1 (left) and Mia-PaCa-2 (right) transfected with indicated siRNA-expressing vectors to TTLL4 (si#466 and si#2692, and si#2146) and a negative control vector (siEGFP). Each average is plotted with error bars indicating SD (standard deviation) after 6 days incubation with Geneticin. “ABS” on Y-axis means absorbance at 490 nm, and at 630 nm as reference, measured with a microplate reader. These experiments were carried out in triplicate. Transfected with si#466 and si#2692 in Panc-1 (left) and Mia-PaCa-2 (right) resulted in a drastic reduction in the number of viable cells, compared with si#2146 and siEGFP for which no knockdown effect was observed (P<0.001, Student's t-test).

FIG. 3 shows that the TTLL4 over-expression promoted cell growth. MTT assay evaluated the cell growth 48 hours after transfection with wild-type TTLL4, enzyme-dead TTLL4 (E906A), or mock, in COS7 cells. (A) Western blot analysis confirmed wild-type TTLL4 and enzyme-dead TTLL4 (E906A) expressions. (B) Western blot analysis using GT 335 antibody indicated that wild-type TTLL4 over-expression enhanced polyglutamylation, while the enzyme-dead TTLL4 (E906A) expressions did not. (C) MTT assay 48 hours after transfection with wild-type TTLL4, enzyme-dead TTLL4 (E906A), or mock in COS-7 cells showed that wild-type TTLL4 promoted cell growth significantly (P<0.001, Student's t-test), but not enzyme-dead TTLL4 (E906A), comparing with the growth of mock-transfected cells.

FIG. 4 shows that TTLL4 polyglutamylates a none-tubulin protein, PELP1. (A) GT335 antibody detected the increased level of polyglutamylation in several proteins, including alpha-tubulin and beta-tubulin around 60 kDa-band, when TTLL4 was over-expressed in Hela cells. (B) GT335 antibody detected several endogenous polyglutamylated proteins in KLM-1 (left) and Mia-PaCa-2 (right) cell lines. When TTLL4 expression was knocked down KLM-1 (left) and Mia-PaCa-2 (right) cell lines, only 200 kDa-band was diminished commonly in both cell lines, which was also detected in over-expression of TTLL4 as shown in FIG. 4A. Among the candidate polyglutamylated proteins identified by the proteomic approach (van Dijk et al JBC 2007), this 200 kDa-band was likely to correspond to PELP1 (prolin-glutamic acid-leucine-rich protein 1). (C) To confirm the polyglutamylation of PELP1 by TTLL4, PELP1 was immunoprecipitated from the cell lysates when TTLL4 was over-expressed in Hela cells, and polyglutamylated PELP1 was detected by GT335 antibody. Polyglutamylation level of PELP1 was clearly increased when TTLL4 was over-expressed. (D) When TTLL4 was knocked down in KLM-1 cells (left) and Mia-Paca-2 cells (right), western blot analysis by using GT335 antibody followed by immunoprecipitation by anti-PELP1 antibody demonstrated that polyglutamylation level of PELPI was decreased.

FIG. 5 shows the constructs of PELP1 designed for co-transformation with TTLL4. (A) PELP1 has several functional domains interacting with several signaling molecules. PELP1 has a highly glutamate-rich region between codon 887 and codon 964. (B) The full-length and three deletion constructs of PELP (del.887-954, del.887-, and del.1003-) were designed, lacking the glutamate-rich region (codon 887-964) or the C-terminal region (codon 887-, codon 1003-), respectively. Each of PELP1 constructs was co-transfected with wild-type TTLL4 or enzyme-dead TTLL4 (E906A).

FIG. 6 shows that TTLL4 polyglutamylates the glutamate-rich stretch region of PELP1. (A) The full-length PELP1(Wt) and two deletion constructs (del.887-954, del.887-) were co-transfeted with wild-type TTLL4 or enzyme-dead TTLL4 (E906A) in o HeLa cells. GT335 detected the polyglutamylated PELP1 when The full-length PELP1 was co-transfected with wild-type TTLL4, but not in the two deletion constructs lacking the glutamate-rich region. (B) The full-length PELP1 and three deletion constructs (del.887-954, del.887-, and del.1003-) were co-transfected with wild-type TTLL4 or enzyme-dead TTLL4 (E906A) into HeLa cells. GT335 antibody detected the polyglutamylated PELP1 when the full-length PELP1 or PELP1 del. 1003- were cotransfected with wild-type TTLL4, but not in the two deletion PELP1 constructs lacking the glutamate-rich region (del.887-954, del.887-). Immunoblot using anti-HA antibody showed the expression of each of PELP1 constructs and immunoblot using anti-Flag antibody showed TTLL4 or mutant TTLL4 expression.

FIG. 7 shows an interaction of PELP1 and histone H3. (A) Interaction of PELP1 and histone H3 by immunoprecipitation. Western blotting using anti-histone H3 antibody indicated that histone H3 was co-immunoprecipitated with PELP1 in PDAC cell. (B) The interaction of PELP with histone H3 was diminished when TTLL4 was knocked down. (C) Acetylation level of histone H3 was increased in TTLL4 knockdown (siTTLL4), compared with control (siEGFP).

FIG. 8 shows an interaction of PELP1 with LAS1L, SENP3 and TEX10. LAS1L, SENP3 and TEX10 were identified as candidates of PELP1-interacting proteins by mass spectrometric analysis following immunoprecipitation. (A) PELP1-HA vector and/or LAS1L-Flag vector were co-transfected into COS-7 cells. Protein complex containing PELP1-HA and/or LAS1L-Flag were immunoprecipitated from the cell extracts by anti-HA antibody (middle) or anti-Flag antibody (lower), respectively. Western blotting using anti-HA antibody indicated that PELP1-HA was co-immunoprecipitated with LAS1L-Flag when the both expression vectors were co-transfected (left). Western blotting using anti-Flag antibody indicated that LAS1L-Flag was co-immunoprecipitated with PELP1-HA when the both expression vectors were co-expressed (right). (B) PELP1-HA vector and/or SENP3-Flag vector were co-transfected into COS-7 cells, and a protein complex containing PELP1-HA and/or SENP3-Flag was immunoprecipitated from the cell extracts by anti-HA antibody (middle) or anti-Flag antibody (lower), respectively. (C) PELP1-HA vector and/or TEX10-Flag vector were co-transfected into COS-7 cells, and a protein complex containing PELP1-HA and/or TEX10-Flag was immunoprecipitated from the cell extracts by anti-HA antibody (middle) or anti-Flag antibody (lower), respectively.

FIG. 9 shows that the effect of over-expression or knock douwn of TTLL4 on the interaction of PELP1 with LAS1L or SENP3. (A) The affinity of PELP1 and LAS1L is increasing when TTLL4 was over-expressed (upper), and the affinity of PELP1 and LAS1L is decreasing when TTLL4 was knocked down (lower). (B) The affinity of PELP1 and SENP3 is decreasing when TTLL4 was over-expressed (upper), and the affinity of PELP1 and LAS1L is increasing when TTLL4 was knocked down (lower).

### DESCRIPTION OF EMBODIMENTS

### Definitions

The words “a”, “an”, and “the” as used herein mean “at least one” unless otherwise specifically indicated.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or polynucleotides.

The terms “polynucleotide”, “oligonucleotide” “nucleotide”, “nucleic acid”, and “nucleic acid molecule” are used interchangeably herein to refer to a polymer of nucleic acid residues and, unless otherwise specifically indicated are referred to by their commonly accepted single-letter codes. The terms apply to nucleic acid (nucleotide) polymers in which one or more nucleic acids are linked by ester bonding. The nucleic acid polymers may be composed of DNA, RNA or a combination thereof and encompass both naturally-occurring and non-naturally occurring nucleic acid polymers.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

Unless otherwise defined, all technical and scientific terms used herein have the same meaning as commonly understood by one of ordinary skill in the art to which this invention belongs. In case of conflict, the present specification, including definitions, will control.

Genes or Proteins

The present invention relates to TTLL4 (tubulin tyrosine ligase-like family, member 4), PELP1 (proline, glutamate and leucine rich protein 1), LAS1L (LAS1-like) and SENP3 (SUMO1/sentrin/SMT3 specific protease 3), and these polynucleotides or polypeptides are utilized in the methods, kits, compositions or the like which are provided by the present invention. The nucleic acid and amino acid sequences of TTLL4, PELP1, LAS1L and SENP3 are known to those skilled in the art, and can be obtained, for example, from gene databases on the web site such as GenBank™.

An exemplified nucleic acid sequence of the TTLL4 gene is shown in SEQ ID NO: 18 (GenBank accession No. NM_{—}014640), and an exemplified amino acid sequence of the TTLL4 polypeptide is shown in SEQ ID NO: 19 and (GenBank accession No. NP_{—}055455.3), but not limited to those. An exemplified nucleic acid sequence of the PELP1 gene is shown in SEQ ID NO: 20 (GenBank accession No. NM_{—}014389.2), and an exemplified amino acid sequence of the TTLL4 polypeptide is shown in SEQ ID NO: 19 and (GenBank accession No. NP_{—}055204.2), but not limited to those. An exemplified nucleic acid sequence of the LAS1L gene is shown in SEQ ID NO: 28 (GenBank accession No. NM_{—}031206.3), and an exemplified amino acid sequence of the LAS1L polypeptide is shown in SEQ ID NO: 29 and (GenBank accession No. NP_{—} 112483.1), but not limited to those. An exemplified nucleic acid sequence of the SENP3 gene is shown in SEQ ID NO: 30 (GenBank accession No. NM_{—}015670.4), and an exemplified amino acid sequence of the TTLL4 polypeptide is shown in SEQ ID NO: 31 and (GenBank accession No. NP_{—}056485.2), but not limited to those.

According to an aspect of the present invention, functional equivalents are also considered to be included in the definition of “polypeptides” of the present invention (i.e., TTLL4 polypeptides, PELP1 polypeptides, LAS1L polypeptides or SENP3 polypeptides). Herein, a “functional equivalent” of a protein (e.g., a TTLL4 polypeptide) is a polypeptide that has a biological activity equivalent to the protein. Namely, any polypeptide that retains the biological ability (e.g., any of the biological activities of TTLL4 shown below) may be used as such a functional equivalent in the present invention. Such functional equivalents include those wherein one or more amino acids are substituted, deleted, added, or inserted to the natural occurring amino acid sequence of the protein. Alternatively, the polypeptide may be composed an amino acid sequence having at least about 80% homology (also referred to as sequence identity) to the sequence of the respective protein, more preferably at least about 90% to 95% homology, often about 96%, 97%, 98% or 99% homology. In other embodiments, the polypeptide can be encoded by a polynucleotide that hybridizes under stringent conditions to the natural occurring nucleotide sequence of the gene.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to that of the human protein of the present invention (i.e., a TTLL4 polypeptide, PELP1 polypeptide, LAS1L polypeptide or SENP3 polypeptide), it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a condition of hybridization for isolating a DNA encoding a polypeptide functionally equivalent to the above human TTLL4, PELP1, LAS1L or SENP3 protein can be routinely selected by a person skilled in the art. For example, hybridization may be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42 degrees C., 2×SSC, 0.1% SDS, preferably 50 degrees C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

Generally, it is known that modifications of one or more amino acid in a protein do not influence the function of the protein. In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity of the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or fewer, preferably 20 amino acids or fewer, more preferably 10 amino acids or fewer, more preferably 6 amino acids or fewer, and even more preferably 3 amino acids or fewer.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

### 1) Alanine (A), Glycine (G);

2) Aspartic acid (D), Glutamic acid (E);

### 3) Aspargine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cystein (C), Methionine (M) (see, e.g., Creighton, Proteins 1984).
Such conservatively modified polypeptides are included in the present protein. However, the present invention is not restricted thereto and the protein includes non-conservative modifications, so long as at least one biological activity of the protein is retained. Furthermore, the modified proteins do not exclude polymorphic variants, interspecies homologues, and those encoded by alleles of these proteins.

Moreover, in the context of the present invention, the genes or polynucleotides (i.e., TTLL4 polynucleotides, PELP1 polynucleotides, LAS1L polynucleotides or SENP3 polynucleotides) encompass polynucleotides that encode such functional equivalents of the proteins (i.e., TTLL4 polypeptides, PELP1 polypeptides, LAS1L polypeptides or SENP3 polypeptides). In addition to hybridization, a gene amplification method, for example, the polymerase chain reaction (PCR) method, can be utilized to isolate a polynucleotide encoding a polypeptide functionally equivalent to the protein, using a primer synthesized based on the sequence above information. Polynucleotides and polypeptides that are functionally equivalent to the human gene and protein, respectively, normally have a high homology to the originating nucleotide or amino acid sequence thereof. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

Double-Stranded Molecules:

As used herein, the term “isolated double-stranded molecule” refers to a nucleic acid molecule that inhibits expression of a target gene and includes, for example, short interfering RNA (siRNA; e.g., double-stranded ribonucleic acid (dsRNA) or small hairpin RNA (shRNA)) and short interfering DNA/RNA (siD/R-NA; e.g., double-stranded chimera of DNA and RNA (dsD/R-NA) or small hairpin chimera of DNA and RNA (shD/R-NA)).

As used herein, the term “siRNA” refers to a double-stranded RNA molecule which prevents translation of a target mRNA. Standard techniques of introducing siRNA into the cell are used, including those in which DNA is a template from which RNA is transcribed. The siRNA includes a TTLL4 sense nucleic acid sequence (also referred to as “sense strand”), a TTLL4 antisense nucleic acid sequence (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules composed of complementary sequences to one another and that have annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, composed of first and second regions complementary to one another, i.e., sense and antisense strands. The degree of complementarity and orientation of the regions is sufficient such that base pairing occurs between the regions, the first and second regions are joined by a loop region, the loop results from a lack of base pairing between nucleotides (or nucleotide analogs) within the loop region. The loop region of an shRNA is a single-stranded region intervening between the sense and antisense strands and may also be referred to as “intervening single-strand”.

As used herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a TTLL4 sense nucleic acid sequence (also referred to as “sense strand”), a TTLL4 antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules composed of complementary sequences to one another and that have annealed together via the complementary sequences to form a double-stranded polynucleotide molecule. The nucleotide sequence of two strands may include not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, composed of a first and second regions complementary to one another, i.e., sense and antisense strands. The degree of complementarity and orientation of the regions is sufficient such that base pairing occurs between the regions, the first and second regions are joined by a loop region, the loop results from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, an “isolated nucleic acid” is a nucleic acid removed from its original environment (e.g., the natural environment if naturally occurring) and thus, synthetically altered from its natural state. In the present invention, examples of isolated nucleic acid includes DNA, RNA, and derivatives thereof.

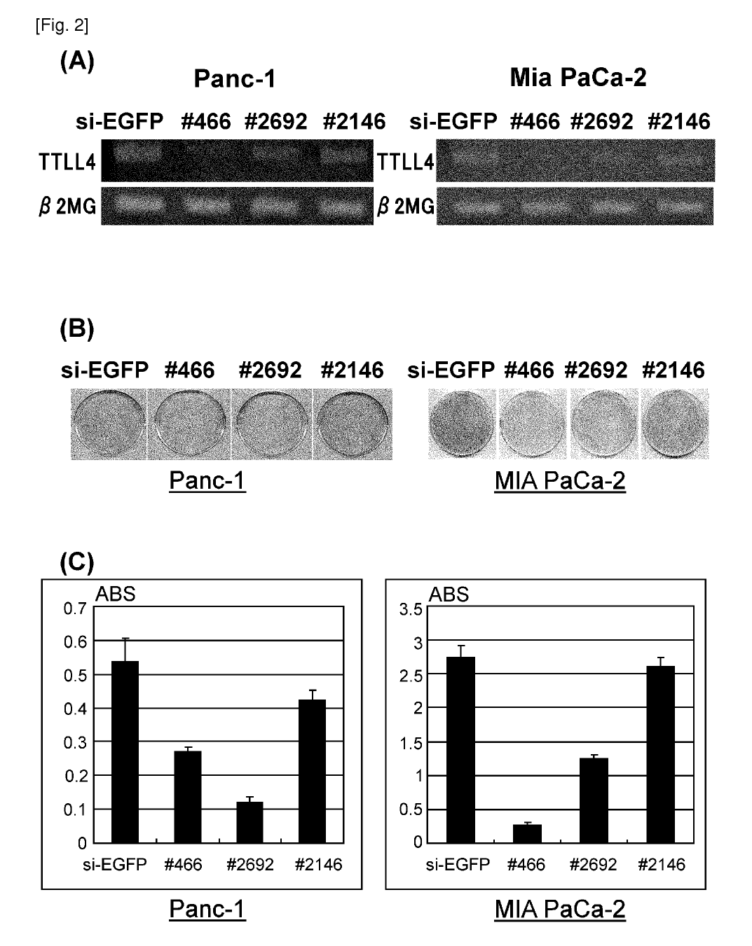

A double-stranded molecule against TTLL4, which molecule hybridizes to target mRNA, decreases or inhibits production of the protein encoded by TTLL4 gene by associating with the normally single-stranded mRNA transcript of the gene, thereby interfering with translation and thus, inhibiting expression of the protein. As demonstrated herein, the expression of TTLL4 in PDAC cell lines was inhibited by dsRNAs (FIG. 2). Therefore the present invention provides isolated double-stranded molecules that are capable of inhibiting the expression of TTLL4 gene when introduced into a cell expressing the gene. The target sequence of the double-stranded molecules may be designed by an siRNA design algorithm such as that mentioned below.

TTLL4 target sequence includes, for example, nucleotides

SEQ ID NO: 7 (at the position 466-485nt of SEQ ID NO: 18)

SEQ ID NO: 8 (at the position 2692-2710nt of SEQ ID NO: 18)

Specifically, the present invention provides the following double-stranded molecules [1] to [18]:

[1] An isolated double-stranded molecule that, when introduced into a cell, inhibits in vivo expression of TTLL4 and cell proliferation, such molecules composed of a sense strand and an antisense strand complementary thereto, hybridized to each other to form the double-stranded molecule;

[2] The double-stranded molecule of [1], wherein said double-stranded molecule acts on mRNA, matching a target sequence selected from among SEQ ID NO: 7 (at the position of 466-485nt of SEQ ID NO: 18), SEQ ID NO: 8 (at the position of 2692-2710nt of SEQ ID NO: 18)

[3] The double-stranded molecule of [2], wherein the sense strand contains a sequence corresponding to a target sequence selected from among SEQ ID NOs: 7 and 8;
[4] The double-stranded molecule of [3], having a length of less than about 100 nucleotides;

[5] The double-stranded molecule of [4], having a length of less than about 75 nucleotides;

[6] The double-stranded molecule of [5], having a length of less than about 50 nucleotides;

[7] The double-stranded molecule of [6], having a length of less than about 25 nucleotides;

[8] The double-stranded molecule of [7], having a length of between about 19 and about 25 nucleotides;

[9] The double-stranded molecule of [1], composed of a single polynucleotide having both the sense and antisense strands linked by an intervening single-strand;

[10] The double-stranded molecule of [9], having the general formula 5′-[A]-[B]-[A′]-3′, wherein [A] is the sense strand containing a sequence corresponding to a target sequence selected from among SEQ ID NOs: 7 and 8, [B] is the intervening single-strand composed of 3 to 23 nucleotides, and [A′] is the antisense strand containing a sequence complementary to [A];

[11] The double-stranded molecule of [1], composed of RNA;

[12] The double-stranded molecule of [1], composed of both DNA and RNA;

[13] The double-stranded molecule of [12], wherein the molecule is a hybrid of a DNA polynucleotide and an RNA polynucleotide;

[14] The double-stranded molecule of [13] wherein the sense and the antisense strands are composed of DNA and RNA, respectively;

[15] The double-stranded molecule of [12], wherein the molecule is a chimera of DNA and RNA;

[16] The double-stranded molecule of [15], wherein a region flanking to the 3′-end of the antisense strand, or both of a region flanking to the 5′-end of sense strand and a region flanking to the 3′-end of antisense strand are RNA;

[17] The double-stranded molecule of [16], wherein the flanking region is composed of 9 to 13 nucleotides; and

[18] The double-stranded molecule of [2], wherein the molecule contains 3′ overhang;

The double-stranded molecule of the present invention will be described in more detail below.

Methods for designing double-stranded molecules having the ability to inhibit target gene expression in cells are known (See, for example, U.S. Pat. No. 6,506,559, herein incorporated by reference in its entirety). For example, a computer program for designing siRNAs is available from the Ambion website (www.ambion.com/techlib/misc/siRNA_finder.html).

The computer program selects target nucleotide sequences for double-stranded molecules based on the following protocol.

Selection of Target Sites:

1. Beginning with the AUG start codon of the transcript, scan downstream for AA di-nucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: ncbi.nlm.nih.gov/BLAST/, is used (Altschul S F et al., Nucleic Acids Res 1997 Sep. 1, 25(17): 3389-402).

3. Select qualifying target sequences for synthesis. Selecting several target sequences along the length of the gene to evaluate is typical.

Using the above protocol, the target sequence of the isolated double-stranded molecules of the present invention were designed as SEQ ID NOs: 7 and 8 for TTLL4 gene.

Double-stranded molecules targeting the above-mentioned target sequences were respectively examined for their ability to suppress the growth of cells expressing the target genes. Therefore, the present invention provides double-stranded molecules targeting any of the sequences selected from the group of

SEQ ID NO: 7 (at the position 466-485nt of SEQ ID NO: 18); and SEQ ID NO: 8 (at the position 2692-2710nt of SEQ ID NO: 18) for TTLL4 gene.

The double-stranded molecule of the present invention may be directed to a single target TTLL4 gene sequence or may be directed to a plurality of target TTLL4 gene sequences.

The double-stranded molecules of the present invention targeting the above-mentioned targeting sequence of TTLL4 gene include isolated polynucleotides that contain any of the nucleic acid sequences of target sequences and/or complementary sequences to the target sequences. Examples of polynucleotides targeting TTLL4 gene include those containing the sequence of SEQ ID NO: 7 or 8 and/or complementary sequences to these nucleotides. However, the present invention is not limited to these examples, and minor modifications in the aforementioned nucleic acid sequences are acceptable so long as the modified molecule retains the ability to suppress the expression of TTLL4 gene. Herein, the phrase “minor modification” as used in connection with a nucleic acid sequence indicates one, two or several substitution, deletion, addition or insertion of nucleic acids to the sequence.

In the context of the present invention, the term “several” as applies to nucleic acid substitutions, deletions, additions and/or insertions may mean 3-7, preferably 3-5, more preferably 3-4, even more preferably 3 nucleic acid residues.

According to the present invention, a double-stranded molecule of the present invention can be tested for its ability using the methods utilized in the Examples. In the Examples herein below, double-stranded molecules composed of sense strands of various portions of mRNA of TTLL4 genes or antisense strands complementary thereto were tested in vitro for their ability to decrease production of TTLL4 gene product in pancreatic cancer cell lines (e.g., using Panc-1, Mia-PaCa-2 for TTLL4) according to standard methods. Furthermore, for example, reduction in TTLL4 gene product in cells contacted with the candidate double-stranded molecule compared to cells cultured in the absence of the candidate molecule can be detected by, e.g., RT-PCR using primers for TTLL4 mRNA mentioned under Example 1 item “Semi-quantitative RT-PCR”. Sequences which decrease the production of TTLL4 gene product in in vitro cell-based assays can then be tested for there inhibitory effects on cell growth. Sequences which inhibit cell growth in in vitro cell-based assay can then be tested for their in vivo ability using animals with cancer, e.g., nude mouse xenograft models, to confirm decreased production of TTLL4 product and decreased cancer cell growth.

When the isolated polynucleotide is RNA or a derivative thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides may also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The polynucleotide is preferably less than 4207 nucleotides in length for TTLL4. For example, the polynucleotide is less than 500, 200, 100, 75, 50, or 25 nucleotides in length for all of the genes. The isolated polynucleotides of the present invention are useful for forming double-stranded molecules against NM_{—}014640 gene or preparing template DNAs encoding the double-stranded molecules. When the polynucleotides are used for forming double-stranded molecules, the polynucleotide may be longer than 19 nucleotides, preferably longer than 21 nucleotides, and more preferably has a length of between about 19 and 25 nucleotides. Alternatively, the double-stranded molecules of the present invention may be double-stranded molecules, wherein the sense strand hybridizes with antisense strand at the target sequence to form the double-stranded molecule having less than 500, 200, 100, 75, 50 or 25 nucleotides pair in length. Preferably, the double-stranded molecules have between about 19 and about 25 nucleotides pair in length. Further, the sense strand of the double-stranded molecule may preferably include less than 500, 200, 100, 75, 50, 30, 27 or 25 nucleotides, more preferably, between about 19 and about 25 or 27 nucleotides.

The double-stranded molecules of the present invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include, but are not limited to, phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C— methyl nucleotides, and inverted deoxybasic residue incorporation (US20060122137).

In another embodiment, modifications can be used to enhance the stability or to increase targeting efficiency of the double-stranded molecule. Examples of such modifications include, but are not limited to, chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine.

In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule composed of a DNA strand (polynucleotide) and an RNA strand (polynucleotide), a chimera type double-stranded molecule containing both DNA and RNA on any or both of the single strands (polynucleotides), or the like may be formed for enhancing stability of the double-stranded molecule.

The hybrid of a DNA strand and an RNA strand may be either where the sense strand is DNA and the antisense strand is RNA, or the opposite so long as it can inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule preferably contains as much DNA as possible, whereas to induce inhibition of the target gene expression, the molecule is required to be RNA within a range to induce sufficient inhibition of the expression.

As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in preferable embodiments, a region flanking to the 3′-end of the antisense strand, or both of a region flanking to the 5′-end of sense strand and a region flanking to the 3′-end of antisense strand are composed of RNA. For instance, the chimera or hybrid type double-stranded molecule of the present invention include following combinations.

sense strand:

5′-[---DNA---]-3′

3′-(RNA)-[DNA]-5′

: antisense strand,

sense strand:

5′-(RNA)-[DNA]-3′

3′-(RNA)-[DNA]-5′

: antisense strand, and

sense strand:

5′-(RNA)-[DNA]-3′

3′-(---RNA---)-5′

: antisense strand.

The upstream partial region preferably is a domain composed of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA includes the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence composed of an arbitrary nucleotide sequence can be located between the sense and antisense sequence in order to form the hairpin loop structure. Thus, the present invention also provides a double-stranded molecule having the general formula 5′-[A]-[B]-[A′]-3′, wherein [A] is the sense strand containing a sequence corresponding to a target sequence, [B] is an intervening single-strand and [A′] is the antisense strand containing a complementary sequence to [A]. The target sequence may be selected from among, for example, nucleotides of SEQ ID NO: 7 or 8 for TTLL4.

The present invention is not limited to these examples, and the target sequence in [A] may be modified sequences from these examples so long as the double-stranded molecule retains the ability to suppress the expression of the targeted TTLL4 gene. The region [A] hybridizes to [A′] to form a loop composed of the region [B]. The intervening single-stranded portion [B], i.e., loop sequence may be preferably 3 to 23 nucleotides in length. The loop sequence, for example, can be selected from among the following sequences (www.ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn D M et al., Nat Rev Mol Cell Biol 2003 June, 4(6): 457-67.

Examples of preferred double-stranded molecules of the present invention having hairpin loop structure are shown below. In the following structure, the loop sequence can be selected from among AUG, CCC, UUCG, CCACC, CTCGAG, AAGCUU, CCACACC, and UUCAAGAGA; however, the present invention is not limited thereto:

(for target sequence SEQ ID NO: 7)
GAAGCAAGUGGAGACACUG-[B]-CAGUGUCUCCACUUGCUUC;
(for target sequence SEQ ID NO: 8)
GAGCCUUGGCAAUAAGUUC-[B]-GAACUUAUUGCCAAGGCUC;

Furthermore, in order to enhance the inhibition activity of the double-stranded molecules, 3′ overhangs may be added to 3′ end of the sense strand and/or the antisense strand of the target sequence. The preferred examples of nucleotides constituting a 3′ overhang include “t” and “u”, but are not limited to. The number of nucleotides to be added is usually 2, but is not limited to, may be more than 2, for example, 3, 4, 5 or more. 3′ overhangs form single strand at the 3′ end of the sense strand and/or the antisense strand of the double-stranded molecule. In cases where double-stranded molecules consists of a single polynucleotide to form a hairpin loop structure, a 3′ overhang sequence may be added to the 3′ end of the single polynucleotide.

The method for preparing the double-stranded molecule is not particularly limited though it is preferable to use a chemical synthetic method known in the art. According to the chemical synthesis method, sense and antisense single-stranded polynucleotides are separately synthesized and then annealed together via an appropriate method to obtain a double-stranded molecule. Specific example for the annealing includes wherein the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking TTLL4 sequences may be identical or different, such that their expression can be modulated independently, or in a temporal or spatial manner. The double-stranded molecules can be transcribed intracellularly by cloning TTLL4 gene templates into a vector containing, e.g., a RNA pol III transcription unit from the small nuclear RNA (snRNA) U6 or the human H1 RNA promoter.

Vectors Containing a Double-Stranded Molecule of the Present Invention:

Also included in the present invention are vectors containing one or more of the double-stranded molecules described herein, and a cell containing such a vector.

Specifically, the present invention provides the following vector of [1] to [10].

[1] A vector, encoding a double-stranded molecule that, when introduced into a cell, inhibits in vivo expression of TTLL4 and cell proliferation, such molecules composed of a sense strand and an antisense strand complementary thereto, hybridized to each other to form the double-stranded molecule.

[2] The vector of [1], encoding the double-stranded molecule acts on mRNA, matching a target sequence selected from among SEQ ID NO: 7 (at the position of 466-485nt of SEQ ID NO: 18) and SEQ ID NO: 8 (at the position of 2692-2710nt of SEQ ID NO: 18);

[3] The vector of [1], wherein the sense strand contains a sequence corresponding to a target sequence selected from among SEQ ID NOs: 7 and 8;

[4] The vector of [3], wherein the double-stranded molecule hybridizes with antisense strand at the target sequence to form the double-stranded molecule encoding the double-stranded molecule having a length of less than about 100 nucleotides;

[5] The vector of [4], wherein the double-stranded molecule hybridizes with antisense strand at the target sequence to form the double-stranded molecule encoding the double-stranded molecule having a length of less than about 75 nucleotides;

[6] The vector of [5], wherein the double-stranded molecule hybridizes with antisense strand at the target sequence to form the double-stranded molecule encoding the double-stranded molecule having a length of less than about 50 nucleotides;

[7] The vector of [6] wherein the double-stranded molecule hybridizes with antisense strand at the target sequence to form the double-stranded molecule encoding the double-stranded molecule having a length of less than about 25 nucleotides;

[8] The vector of [7], wherein the double-stranded molecule hybridizes with antisense strand at the target sequence to form the double-stranded molecule encoding the double-stranded molecule having a length of between about 19 and about 25 nucleotides;

[9] The vector of [1], wherein the double-stranded molecule is composed of a single polynucleotide having both the sense and antisense strands linked by an intervening single-strand;

[10] The vector of [9], encoding the double-stranded molecule having the general formula 5′-[A]-[B]-[A′]-3′, wherein [A] is the sense strand containing a sequence corresponding to a target sequence selected from among SEQ ID NOs: 7 and 8, [B] is the intervening single-strand composed of 3 to 23 nucleotides, and [A′] is the antisense strand containing a sequence complementary to [A];

A vector of the present invention preferably encodes a double-stranded molecule of the present invention in an expressible form. Herein, the phrase “in an expressible form” indicates that the vector, when introduced into a cell, will express the molecule. In a preferred embodiment, the vector includes regulatory elements necessary for expression of the double-stranded molecule. Such vectors of the present invention may be used for producing the present double-stranded molecules, or directly as an active ingredient for treating cancer.

Vectors of the present invention can be produced, for example, by cloning TTLL4 sequence into an expression vector so that regulatory sequences are operatively-linked to TTLL4 sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vector constructs respectively encoding the sense and antisense strands of the double-stranded molecule are utilized to respectively express the sense and anti-sense strands and then forming a double-stranded molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Methods of Inhibiting or Reducing Growth of a Cancer Cell and Treating Cancer Using a Double-Stranded Molecule of the Present Invention:

In the present invention, three different dsRNAs against TTLL4 were tested for their ability to inhibit cell growth. Among those three dsRNAs, the two dsRNAs, effectively knocked down the expression of the gene in pancreatic cancer cell lines coincided with suppression of cell proliferation (FIGS. 2B and C). The results demonstrate that TTLL4 is involved in cancer cell survival and/or growth, and suppressing the expression of TTLL4 lead to inhibit cancer cell growth, and therefore, the double-stranded molecules against TTLL4 gene may be useful for treating and/or preventing cancer.

Therefore, the present invention provides methods for inhibiting cell growth, i.e., cancer cell growth, by inducing dysfunction of TTLL4 gene via inhibiting the expression of TTLL4. TTLL4 gene expression can be inhibited by any of the aforementioned double-stranded molecules of the present invention which specifically target TTLL4 gene or the vectors of the present invention that can express any of the double-stranded molecules.

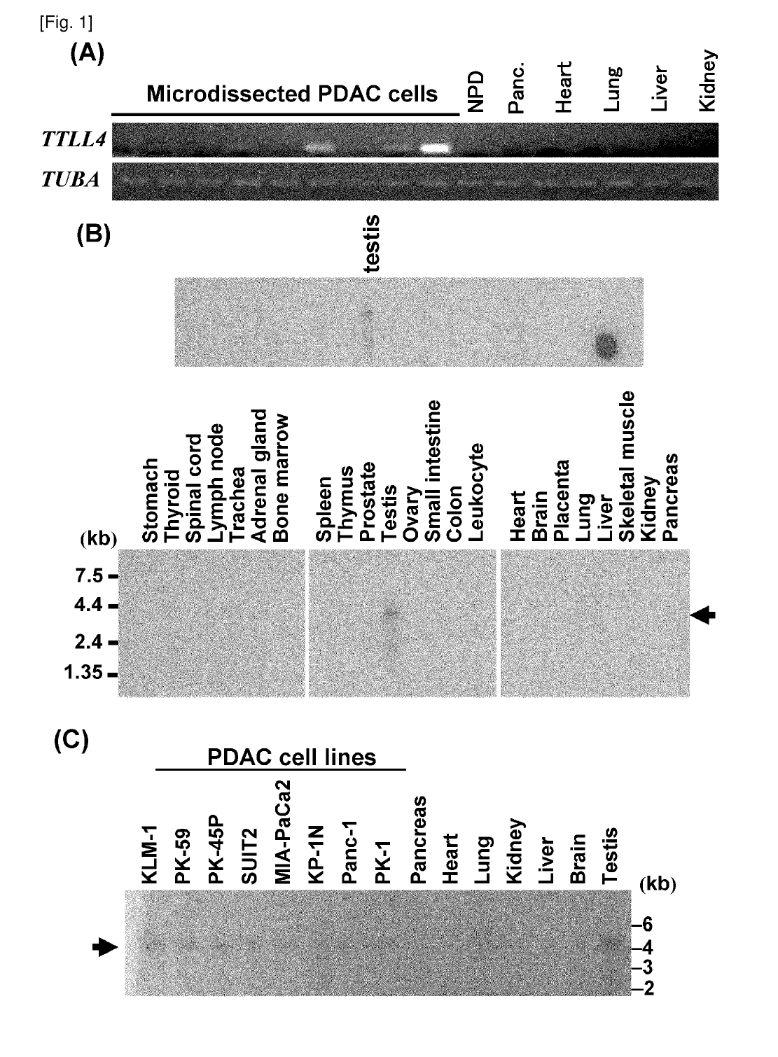

Such ability of the present double-stranded molecules and vectors to inhibit cell growth of cancerous cell indicates that they can be used for methods for treating cancer. Thus, the present invention provides methods to treat patients with cancer by administering a double-stranded molecule against TTLL4 gene or a vector expressing the molecule without adverse effect because that genes were hardly detected in normal organs (FIG. 1).

Specifically, the present invention provides the following methods [1] to [36]:

[1] A method for inhibiting a growth of cancer cell and treating a cancer, wherein the cancer cell or the cancer expresses TTLL4 gene, which method includes the step of ad-ministering at least one isolated double-stranded molecule inhibiting the expression of TTLL4 in a cell over-expressing the gene and the cell proliferation, wherein the double-stranded molecule is composed of a sense strand and an antisense strand complementary thereto, hybridized to each other to form the double-stranded molecule.

[2] The method of [1], wherein the double-stranded molecule acts at mRNA which matches a target sequence selected from among SEQ ID NO: 7 (at the position of 466-485nt of SEQ ID NO: 18) and SEQ ID NO: 8 (at the position of 2692-2710nt of SEQ ID NO: 18).

[3] The method of [2], wherein the sense strand contains the sequence corresponding to a target sequence selected from among SEQ ID NOs: 7 and 8.

[4] The method of [1], wherein the cancer to be treated is pancreatic cancer;

[5] The method of [4], wherein the pancreatic cancer is PDAC;

[6] The method of [1], wherein plural kinds of the double-stranded molecules are ad-ministered;

[7] The method of [3], wherein the double-stranded molecule has a length of less than about 100 nucleotides;

[8] The method of [7], wherein the double-stranded molecule has a length of less than about 75 nucleotides;

[9] The method of [8], wherein the double-stranded molecule has a length of less than about 50 nucleotides;

[10] The method of [9], wherein the double-stranded molecule has a length of less than about 25 nucleotides;

[11] The method of [10], wherein the double-stranded molecule has a length of between about 19 and about 25 nucleotides in length;

[12] The method of [1], wherein the double-stranded molecule is composed of a single polynucleotide containing both the sense strand and the antisense strand linked by an intervening single-strand;

[13] The method of [12], wherein the double-stranded molecule has the general formula 5′-[A]-[B]-[A′]-3′, wherein [A] is the sense strand containing a sequence corresponding to a target sequence selected from among SEQ ID NOs: 7 and 8, [B] is the intervening single strand composed of 3 to 23 nucleotides, and [A′] is the antisense strand containing a sequence complementary to [A];

[14] The method of [1], wherein the double-stranded molecule is an RNA;

[15] The method of [1], wherein the double-stranded molecule contains both DNA and RNA;

[16] The method of [15], wherein the double-stranded molecule is a hybrid of a DNA polynucleotide and an RNA polynucleotide;

[17] The method of [16] wherein the sense and antisense strand polynucleotides are composed of DNA and RNA, respectively;

[18] The method of [15], wherein the double-stranded molecule is a chimera of DNA and RNA;

[19] The method of [18], wherein a region flanking to the 3′-end of the antisense strand, or both of a region flanking to the 5′-end of sense strand and a region flanking to the 3′-end of antisense strand are composed of RNA;

[20] The method of [19], wherein the flanking region is composed of 9 to 13 nucleotides;

[21] The method of [1], wherein the double-stranded molecule contains 3′ overhangs;

[22] The method of [1], wherein the double-stranded molecule is contained in a composition which includes, in addition to the molecule, a transfection-enhancing agent and pharmaceutically acceptable carrier;

[23] The method of [1], wherein the double-stranded molecule is encoded by a vector;

[24] The method of [23], wherein the double-stranded molecule encoded by the vector acts at mRNA which matches a target sequence selected from among SEQ ID NO: 7 (at the position of 455-485nt of SEQ ID NO: 18) and SEQ ID NO: 8 (at the position of 2692-2710nt of SEQ ID NO: 18);

[25] The method of [24], wherein the sense strand of the double-stranded molecule encoded by the vector contains the sequence corresponding to a target sequence selected from among SEQ ID NOs: 7 and 8.

[26] The method of [23], wherein the cancer to be treated is pancreatic cancer;

[27] The method of [26], wherein the pancreatic cancer is PDAC;

[28] The method of [23], wherein plural kinds of the double-stranded molecules are ad-ministered;

[29] The method of [25], wherein the double-stranded molecule encoded by the vector has a length of less than about 100 nucleotides;

[30] The method of [29], wherein the double-stranded molecule encoded by the vector has a length of less than about 75 nucleotides;

[31] The method of [30], wherein the double-stranded molecule encoded by the vector has a length of less than about 50 nucleotides;

[32] The method of [31], wherein the double-stranded molecule encoded by the vector has a length of less than about 25 nucleotides;

[33] The method of [32], wherein the double-stranded molecule encoded by the vector has a length of between about 19 and about 25 nucleotides in length;

[34] The method of [23], wherein the double-stranded molecule encoded by the vector is composed of a single polynucleotide containing both the sense strand and the antisense strand linked by an intervening single-strand;

[35] The method of [34], wherein the double-stranded molecule encoded by the vector has the general formula 5′-[A]-[B]-[A′]-3′, wherein [A] is the sense strand containing a sequence corresponding to a target sequence selected from among SEQ ID NOs: 7 and 8, [B] is a intervening single-strand is composed of 3 to 23 nucleotides, and [A′] is the antisense strand containing a sequence complementary to [A]; and

[36] The method of [23], wherein the double-stranded molecule encoded by the vector is contained in a composition which includes, in addition to the molecule, a transfection-enhancing agent and pharmaceutically acceptable carrier.

The method of the present invention will be described in more detail below.

The growth of cells expressing TTLL4 gene may be inhibited by contacting the cells with a double-stranded molecule against TTLL4 gene, a vector expressing the molecule or a composition containing the same. The cell may be further contacted with a transfection agent. Suitable transfection agents are known in the art. The phrase “inhibition of cell growth” indicates that the cell proliferates at a lower rate or has decreased viability as compared to a cell not exposed to the molecule. Cell growth may be measured by methods known in the art, e.g., using the MTT cell proliferation assay.

The growth of any kind of cell may be suppressed according to the present method so long as the cell over-expresses the target gene of the double-stranded molecule of the present invention. Exemplary cells include pancreatic cancer cells, including PDAC.

Thus, patients suffering from or at risk of developing disease related to TTLL4 may be treated by administering at least one of the present double-stranded molecules, at least one vector expressing at least one of the molecules or at least one composition containing at least one of the molecules. For example, patients of pancreatic cancer may be treated according to the present methods. The type of cancer may be identified by standard methods according to the particular type of tumor to be diagnosed. Preferably, patients treated by the methods of the present invention are selected by detecting the expression of TTLL4 in a biopsy from the patient by RT-PCR or immunoassay. More preferably, before the treatment of the present invention, the biopsy specimen from the subject is confirmed for TTLL4 gene over-expression by methods known in the art, for example, immunohistochemical analysis or RT-PCR.

According to the present method to inhibit cell growth and thereby treating cancer, when administering plural kinds of the double-stranded molecules (or vectors ex-pressing or compositions containing the same), each of the molecules may have different structures but acts at mRNA which matches the same target sequence of TTLL4. Alternatively plural kinds of the double-stranded molecules may acts at mRNA which matches different target sequence of same gene. For example, the method may utilize double-stranded molecules directed to one, two or more target sequences selected from TTLL4.

For inhibiting cell growth, a double-stranded molecule of present invention may be directly introduced into the cells in a form to achieve binding of the molecule with corresponding mRNA transcripts. Alternatively, as described above, a DNA encoding the double-stranded molecule may be introduced into cells as a vector. For introducing the double-stranded molecules and vectors into the cells, transfection-enhancing agent, such as FuGENE (Roche diagnostics), Lipofectamine 2000 (Invitrogen), Oligofectamine (Invitrogen), and Nucleofector (Wako pure Chemical), may be employed.

A treatment is deemed “efficacious” if it leads to clinical benefit such as, reduction in expression of TTLL4 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

It is understood that the double-stranded molecule of the invention degrades the TTLL4 mRNA in substoichiometric amounts. Without wishing to be bound by any theory, it is believed that the double-stranded molecule of the present invention causes degradation of the target mRNA in a catalytic manner. Thus, compared to standard cancer therapies, a significantly less of a double-stranded molecule needs to be delivered at or near the site of cancer to exert therapeutic effect.

One skilled in the art can readily determine an effective amount of the double-stranded molecule of the invention to be administered to a given subject, by taking into account factors such as body weight, age, sex, type of disease, symptoms and other conditions of the subject; the route of administration; and whether the administration is regional or systemic. Generally, an effective amount of the double-stranded molecule of the invention is an intercellular concentration at or near the cancer site of from about 1 nanomolar (nM) to about 100 nM, preferably from about 2 nM to about 50 nM, more preferably from about 2.5 nM to about 10 nM. It is contemplated that greater or smaller amounts of the double-stranded molecule can be administered. The precise dosage required for a particular circumstance may be readily and routinely determined by one of skill in the art.

The present methods can be used to inhibit the growth or metastasis of cancer ex-pressing at least one TTLL4; for example pancreatic cancer, especially PDAC. In particular, a double-stranded molecule containing a target sequence of TTLL4 (i.e., SEQ ID NOs: 7 or 8) is particularly preferred for the treatment of pancreatic cancer.

For treating cancer, the double-stranded molecule of the present invention can also be administered to a subject in combination with a pharmaceutical agent different from the double-stranded molecule. Alternatively, the double-stranded molecule of the invention can be administered to a subject in combination with another therapeutic method designed to treat cancer. For example, the double-stranded molecule of the invention can be administered in combination with therapeutic methods currently employed for treating cancer or preventing cancer metastasis (e.g., radiation therapy, surgery and treatment using chemotherapeutic agents, such as cisplatin, carboplatin, cyclophosphamide, 5-fluorouracil, adriamycin, daunorubicin or tamoxifen).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with the present double-stranded molecule include the Minis Transit TKO lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as pancreatic tissue, and can also increase the blood half-life of the double-stranded molecule. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded molecule includes a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

Particularly preferably, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can include both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as poly-acrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH3 and a solvent mixture such as tetrahydrofuran and water in a 30:12 ratio at 60 degrees C.

Vectors expressing a double-stranded molecule of the invention are discussed above. Such vectors expressing at least one double-stranded molecule of the invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecule of the invention can be administered to the subject by any means suitable for delivering the double-stranded molecule into cancer sites. For example, the double-stranded molecule can be administered by gene gun, electro-poration, or by other suitable parenteral or enteral administration routes.

Suitable enteral administration routes include oral, rectal, or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection); subcutaneous injection or de-position including subcutaneous infusion (such as by osmotic pumps); direct application to the area at or near the site of cancer, for example by a catheter or other placement device (e.g., a suppository or an implant including a porous, non-porous, or gelatinous material); and inhalation. It is preferred that injections or infusions of the double-stranded molecule or vector be given at or near the site of cancer.

The double-stranded molecule of the invention can be administered in a single dose or in multiple doses. Where the administration of the double-stranded molecule of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded molecule of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, more preferably from about seven to about ten days. In a preferred dosage regimen, the double-stranded molecule is injected at or near the site of cancer once a day for seven days. Where a dosage regimen includes multiple administrations, it is understood that the effective amount of a double-stranded molecule administered to the subject can include the total amount of a double-stranded molecule administered over the entire dosage regimen.

Compositions Containing a Double-Stranded Molecule of the Present Invention:

In addition to the above, the present invention also provides pharmaceutical compositions that include at least one of the present double-stranded molecules or the vectors coding for the molecules. Specifically, the present invention provides the following compositions [1] to [36]:

[1] A composition for inhibiting a growth of cancer cell and treating a cancer, wherein the cancer cell and the cancer expresses TTLL4 gene, including at least one isolated double-stranded molecule inhibiting the expression of TTLL4 and the cell proliferation, which molecule is composed of a sense strand and an antisense strand complementary thereto, hybridized to each other to form the double-stranded molecule.

[2] The composition of [1], wherein the double-stranded molecule acts at mRNA which matches a target sequence selected from among SEQ ID NO: 7 (at the position of 466-485nt of SEQ ID NO: 18) and SEQ ID NO: 8 (at the position of 2692-2710nt of SEQ ID NO: 18);

[3] The composition of [2], wherein the double-stranded molecule, wherein the sense strand contains a sequence corresponding to a target sequence selected from among SEQ ID NOs: 7 and 8.

[4] The composition of [1], wherein the cancer to be treated is pancreatic cancer;

[5] The composition of [4], wherein the pancreatic cancer is PDAC;

[6] The composition of [1], wherein the composition contains plural kinds of the double-stranded molecules;

[7] The composition of [3], wherein the double-stranded molecule has a length of less than about 100 nucleotides;

[8] The composition of [7], wherein the double-stranded molecule has a length of less than about 75 nucleotides;

[9] The composition of [8], wherein the double-stranded molecule has a length of less than about 50 nucleotides;

[10] The composition of [9], wherein the double-stranded molecule has a length of less than about 25 nucleotides;

[11] The composition of [10], wherein the double-stranded molecule has a length of between about 19 and about 25 nucleotides;

[12] The composition of [1], wherein the double-stranded molecule is composed of a single polynucleotide containing the sense strand and the antisense strand linked by an intervening single-strand;

[13] The composition of [12], wherein the double-stranded molecule has the general formula 5′-[A]-[B]-[A′]-3′, wherein [A] is the sense strand sequence contains a sequence corresponding to a target sequence selected from among SEQ ID NOs: 7 and 8, [B] is the intervening single-strand consisting of 3 to 23 nucleotides, and [A′] is the antisense strand contains a sequence complementary to [A];

[14] The composition of [1], wherein the double-stranded molecule is an RNA;

[15] The composition of [1], wherein the double-stranded molecule is DNA and/or RNA;

[16] The composition of [15], wherein the double-stranded molecule is a hybrid of a DNA polynucleotide and an RNA polynucleotide;

[17] The composition of [16], wherein the sense and antisense strand polynucleotides are composed of DNA and RNA, respectively;

[18] The composition of [15], wherein the double-stranded molecule is a chimera of DNA and RNA;

[19] The composition of [18], wherein a region flanking to the 3′-end of the antisense strand, or both of a region flanking to the 5′-end of sense strand and a region flanking to the 3′-end of antisense strand are composed of RNA;

[20] The composition of [19], wherein the flanking region is composed of 9 to 13 nucleotides;

[21] The composition of [1], wherein the double-stranded molecule contains 3′ overhangs;

[22] The composition of [1], wherein the composition includes a transfection-enhancing agent and pharmaceutically acceptable carrier.

[23] The composition of [1], wherein the double-stranded molecule is encoded by a vector and contained in the composition;

[24] The composition of [23], wherein the double-stranded molecule encoded by the vector acts at mRNA which matches a target sequence selected from among SEQ ID NO: 7 (at the position of 466-485nt of SEQ ID NO: 18) and SEQ ID NO: 8 (at the position of 2692-2710nt of SEQ ID NO: 18).

[25] The composition of [24], wherein the sense strand of the double-stranded molecule encoded by the vector contains the sequence corresponding to a target sequence selected from among SEQ ID NOs: 7 and 8.

[26] The composition of [23], wherein the cancer to be treated is pancreatic cancer;

[27] The composition of [26], wherein the pancreatic cancer is PDAC;

[28] The composition of [23], wherein plural kinds of the double-stranded molecules are administered;

[29] The composition of [25], wherein the double-stranded molecule encoded by the vector has a length of less than about 100 nucleotides;

[30] The composition of [29], wherein the double-stranded molecule encoded by the vector has a length of less than about 75 nucleotides;

[31] The composition of [30], wherein the double-stranded molecule encoded by the vector has a length of less than about 50 nucleotides;

[32] The composition of [31], wherein the double-stranded molecule encoded by the vector has a length of less than about 25 nucleotides;

[33] The composition of [32], wherein the double-stranded molecule encoded by the vector has a length of between about 19 and about 25 nucleotides in length;

[34] The composition of [23], wherein the double-stranded molecule encoded by the vector is composed of a single polynucleotide containing both the sense strand and the antisense strand linked by an intervening single-strand;

[35] The composition of [23], wherein the double-stranded molecule has the general formula 5′-[A]-[B]-[A′]-3′, wherein [A] is the sense strand containing a sequence corresponding to a target sequence selected from among SEQ ID NOs: 7 and 8, [B] is a intervening single-strand composed of 3 to 23 nucleotides, and [A′] is the antisense strand containing a sequence complementary to [A]; and

[36] The composition of [23], wherein the composition includes a transfection-enhancing agent and pharmaceutically acceptable carrier.

Suitable compositions of the present invention are described in additional detail below.

The double-stranded molecules of the present invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example, as described in Remington's Pharmaceutical Science, 17th ed., Mack Publishing Company, Easton, Pa. (1985), the entire disclosure of which is herein incorporated by reference.

The present pharmaceutical formulations contain at least one of the double-stranded molecules or vectors encoding them of the present invention (e.g., 0.1 to 90% by weight), or a physiologically acceptable salt of the molecule, mixed with a physiologically acceptable carrier medium. Preferred physiologically acceptable carrier media are water, buffered water, normal saline, 0.4% saline, 0.3% glycine, hyaluronic acid and the like.

According to the present invention, the composition may contain plural kinds of the double-stranded molecules, each of the molecules may be directed to the same target sequence, or different target sequences of TTLL4. For example, the composition may contain double-stranded molecules directed to TTLL4. Alternatively, for example, the composition may contain double-stranded molecules directed to one, two or more target sequences of TTLL4.

Furthermore, the present composition may contain a vector coding for one or plural double-stranded molecules. For example, the vector may encode one, two or several kinds of the present double-stranded molecules. Alternatively, the present composition may contain plural kinds of vectors, each of the vectors coding for a different double-stranded molecule.

Moreover, the present double-stranded molecules may be contained as liposomes in the present composition. See under the item of “Methods of inhibiting or reducing growth of a cancer cell and treating cancer using a double-stranded molecule of the present invention” for details of liposomes.

Pharmaceutical compositions of the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, preferably 25-75%, of one or more double-stranded molecule of the invention. A pharmaceutical composition for aerosol (inhalational) administration can include 0.01-20% by weight, preferably 1-10% by weight, of one or more double-stranded molecule of the invention en-capsulated in a liposome as described above, and propellant. A carrier can also be included as desired; e.g., lecithin for intranasal delivery.

In addition to the above, the present composition may contain other pharmaceutical active ingredients so long as they do not inhibit the in vivo function of the present double-stranded molecules. For example, the composition may contain chemotherapeutic agents conventionally used for treating cancers.

In another embodiment, the present invention also provides the use of the double-stranded nucleic acid molecules of the present invention in manufacturing a pharmaceutical composition for treating a pancreatic cancer characterized by the expression of TTLL4. For example, the present invention relates to a use of double-stranded nucleic acid molecule inhibiting the expression of TTLL4 gene in a cell, which molecule includes a sense strand and an antisense strand complementary thereto, hybridized to each other to form the double-stranded nucleic acid molecule and targets to a sequence selected from among SEQ ID NOs: 7 and 8, for manufacturing a pharmaceutical composition for treating pancreatic cancer expressing TTLL4.

Alternatively, the present invention further provides a method or process for manufacturing a pharmaceutical composition for treating a pancreatic cancer characterized by the TTLL4 expression, wherein the method or process includes a step for formulating a pharmaceutically or physiologically acceptable carrier with a double-stranded nucleic acid molecule inhibiting the TTLL4 expression in a cell, which over-expresses the gene, which molecule includes a sense strand and an antisense strand complementary thereto, hybridized to each other to form the double-stranded nucleic acid molecule and targets to a sequence selected from among SEQ ID NOs: 7 and 8 as active ingredients.

In another embodiment, the present invention also provides a method or process for manufacturing a pharmaceutical composition for treating a pancreatic cancer characterized by the TTLL4 expression, wherein the method or process includes a step for admixing an active ingredient with a pharmaceutically or physiologically acceptable carrier, wherein the active ingredient is a double-stranded nucleic acid molecule inhibiting the TTLL4 expression in a cell, which over-expresses the gene, which molecule includes a sense strand and an antisense strand complementary thereto, hybridized to each other to form the double-stranded nucleic acid molecule and targets to a sequence selected from among SEQ ID NOs: 7 and 8.

A Method for Diagnosing Pancreatic Cancer:

The expression of TTLL4 was found to be specifically elevated in pancreatic cancer cells (FIG. 1). Therefore, the genes identified herein as well as their transcription and translation products find diagnostic utility as markers for cancer and by measuring the expression of TTLL4 in a cell sample, cancer can be diagnosed or detected by comparing the expression level of TTLL4 between the subject-derived sample with normal sample. Specifically, the present invention provides a method for diagnosing or detecting cancer by determining the expression level of TTLL4 in the subject. Cancers that can be diagnosed by the present method may be preferably pancreatic cancer, more preferably PDAC.

Alternatively, the present invention provides a method for detecting or identifying cancer cells in a subject-derived pancreatic tissue sample, said method including the step of determining the expression level of the TTLL4 gene in a subject-derived biological sample, wherein an increase in said expression level as compared to a normal control level of said gene indicates the presence or suspicion of cancer cells in the tissue.

According to the present invention, an intermediate result for examining the condition of a subject may be provided. Such intermediate result may be combined with additional information to assist a doctor, nurse, or other practitioner to diagnose that a subject suffers from the disease. Alternatively, the present invention may be used to detect cancerous cells in a subject-derived tissue, and provide a doctor with useful information to diagnose that the subject suffers from the disease.

For example, according to the present invention, when there is doubt regarding the presence of cancer cells in the tissue obtained from a subject, clinical decisions can be reached by considering the expression level of the TTLL4 gene, plus a different aspect of the disease including tissue pathology, levels of known tumor marker(s) in blood, and clinical course of the subject, etc. For example, some well-known diagnostic pancreatic cancer markers in blood are BFP, CA19-9, CA72-4, CA125, CA130, CEA, DUPAN-2, IAP, KMO-1, NCC-ST-439, NSE, sICAM-1, SLX, Span-1, STN, TPA, YH-206, elastase I. Namely, in this particular embodiment of the present invention, the outcome of the gene expression analysis serves as an intermediate result for further diagnosis of a subject's disease state.

Specifically, the present invention provides the following methods [1] to [10]:

[1] A method of detecting or diagnosing pancreatic cancer in a subject, including determining a expression level of TTLL4 in the subject derived biological sample, wherein an increase of said level compared to a normal control level of TTLL4 indicates that said subject suffers from or is at risk of developing pancreatic cancer;

[2] The method of [1], wherein said increase is at least 10% greater than said normal control level;

[3] The method of [1], wherein the expression level is detected by a method selected from among:

(a) detecting an mRNA including the sequence of TTLL4,
(b) detecting a protein including the amino acid sequence of TTLL4, and
(c) detecting a biological activity of a protein including the amino acid sequence of TTLL4.

[4] The method of [1], wherein the pancreatic cancer is PDAC;

[5] The method of [3], wherein the expression level is determined by detecting hybridization of a probe to a gene transcript of the gene;

[6] The method of [3], wherein the expression level is determined by detecting the binding of an antibody against the protein encoded by a gene as the expression level of the gene;

[7] The method of [1], wherein the biological sample includes biopsy, sputum, pleural effusion or blood;

[8] The method of [1], wherein the subject-derived biological sample includes an epithelial cell.

[9] The method of [1], wherein the subject-derived biological sample includes a cancer cell.

[10] The method of [1], wherein the subject-derived biological sample includes a cancerous epithelial cell.

The method of diagnosing cancer will be described in more detail below.

A subject to be diagnosed by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the determination so long as it includes the objective transcription or translation product of TTLL4. The biological samples include, but are not limited to, bodily tissues which are desired for diagnosing or are suspicion of suffering from cancer, and fluids, such as biopsy, blood, sputum, pleural effusion and urine. Preferably, the biological sample contains a cell population including an epithelial cell, more preferably a cancerous epithelial cell or an epithelial cell derived from tissue suspected to be cancerous. Further, if necessary, the cell may be purified from the obtained bodily tissues and fluids, and then used as the biological sample.

According to the present invention, the expression level of TTLL4 in the subject-derived biological sample is determined. The expression level can be determined at the transcription (nucleic acid) product level, using methods known in the art. For example, the mRNA of TTLL4 may be quantified using probes by hybridization methods (e.g., Northern hybridization). The detection may be carried out on a chip or an array. The use of an array is preferable for detecting the expression level of a plurality of genes (e.g., various cancer specific genes) including TTLL4. Those skilled in the art can prepare such probes utilizing the sequence information of the TTLL4 (SEQ ID NO 18; GenBank accession number: NM_{—}014640). For example, the cDNA of TTLL4 may be used as the probes. If necessary, the probe may be labeled with a suitable label, such as dyes, fluorescent and isotopes, and the expression level of the gene may be detected as the intensity of the hybridized labels.

Furthermore, the transcription product of TTLL4 may be quantified using primers by amplification-based detection methods (e.g., RT-PCR). Such primers can also be prepared based on the available sequence information of the gene. For example, the primers (SEQ ID NOs: 1, 2, 5 and 6) used in the Example may be employed for the detection by RT-PCR or Northern blot, but the present invention is not restricted thereto.

Specifically, a probe or primer used for the present method hybridizes under stringent, moderately stringent, or low stringent conditions to the mRNA of TTLL4. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the diagnosis of the present invention. For example, the quantity of TTLL4 protein may be determined. A method for determining the quantity of the protein as the translation product includes immunoassay methods that use an antibody specifically recognizing the protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to TTLL4 protein. Methods to prepare these kinds of antibodies for the detection of proteins are well known in the art, and any method may be employed in the present invention to prepare such antibodies and equivalents thereof.

As another method to detect the expression level of TTLL4 gene based on its translation product, the intensity of staining may be observed via immunohistochemical analysis using an antibody against TTLL4 protein. Namely, the observation of strong staining indicates increased presence of the protein and at the same time high expression level of TTLL4 gene.

Alternatively, the translation product may be detected based on its biological activity. Specifically, the TTLL4 protein was demonstrated herein to be involved in the growth of cancer cells. Thus, the cancer cell growth promoting ability of the TTLL4 protein may be used as an index of the TTLL4 protein existing in the biological sample. Alternatively, the polyglutamylation activity may be detected as a biological activity of the TTLL4 protein. For example, the polyglutamylation activity in a sample may be detected by incubating the sample with a substrate to be polyglutamylated and a glutamate as a cofactor. Preferred examples of substrates include polypeptide containing a glutamate rich domain such as the polypeptide corresponding to SEQ ID NO: 23, but are not limited to. More preferably, substrate may be the PELP1 polypeptide or a functional equivalent thereof. After incubation, polyglutamylated substrate by TTLL4 may be detected by an anti-polyglumamylation antibody.

Moreover, in addition to the expression level of TTLL4 gene, the expression level of other cancer-associated genes, for example, genes known to be differentially expressed in pancreatic cancer may also be determined to improve the accuracy of the diagnosis.

The expression level of cancer marker gene including TTLL4 gene in a biological sample can be considered to be increased if it increases from the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of TTLL4 gene in samples from subjects whose disease state are known. Furthermore, the control level can be a database of expression patterns from previously tested cells. Moreover, according to an aspect of the present invention, the expression level of TTLL4 gene in a biological sample may be compared to multiple control levels, which control levels are determined from multiple reference samples. It is preferred to use a control level determined from a reference sample derived from a tissue type similar to that of the patient-derived biological sample. Moreover, it is preferred, to use the standard value of the expression levels of TTLL4 gene in a population with a known disease state. The standard value may be obtained by any method known in the art. For example, a range of mean+/−2 S.D. or mean+/−3 S.D. may be used as standard value.

In the context of the present invention, a control level determined from a biological sample that is known not to be cancerous is referred to as a “normal control level”. On the other hand, if the control level is determined from a cancerous biological sample, it is referred to as a “cancerous control level”.

When the expression level of TTLL4 gene is increased as compared to the normal control level or is similar to the cancerous control level, the subject may be diagnosed to be suffering from or at a risk of developing cancer. Furthermore, in the case where the expression levels of multiple cancer-related genes are compared, a similarity in the gene expression pattern between the sample and the reference which is cancerous indicates that the subject is suffering from or at a risk of developing cancer.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

A Kit for Diagnosing Cancer:

The present invention provides a kit for diagnosing cancer. Preferably, the cancer is pancreatic cancer, more preferably PDAC. Specifically, the kit includes at least one reagent for detecting the expression of the TTLL4 gene in a subject-derived biological sample, which reagent may be selected from the group of:

(a) a reagent for detecting mRNA of the TTLL4 gene;

(b) a reagent for detecting the TTLL4 protein; and

(c) a reagent for detecting the biological activity of the TTLL4 protein.

Suitable reagents for detecting mRNA of the TTLL4 gene include nucleic acids that specifically bind to or identify the TTLL4 mRNA, such as oligonucleotides which have a complementary sequence to a part of the TTLL4 mRNA. These kinds of oligonucleotides are exemplified by primers and probes that are specific to the TTLL4 mRNA. These kinds of oligonucleotides may be prepared based on methods well known in the art. If needed, the reagent for detecting the TTLL4 mRNA may be immobilized on a solid matrix. Moreover, more than one reagent for detecting the TTLL4 mRNA may be included in the kit.

On the other hand, suitable reagents for detecting the TTLL4 protein include antibodies to the TTLL4 protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the TTLL4 protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the TTLL4 protein may be included in the kit.

Furthermore, the biological activity can be determined by, for example, measuring the cell proliferating activity due to the expressed TTLL4 protein in the biological sample. For example, the cell is cultured in the presence of a subject-derived biological sample, and then by detecting the speed of proliferation, or by measuring the cell cycle or the colony forming ability the cell proliferating activity of the biological sample can be determined. If needed, the reagent for detecting the TTLL4 mRNA may be immobilized on a solid matrix. Moreover, more than one reagent for detecting the biological activity of the TTLL4 protein may be included in the kit. For example, the biological activity may be determined by measuring the polglutamylation activity. Accordingly, the reagent for detecting the biological activity may be include a substrate to be polyglutamylated, a glutamate as a cofactor and a reagent for detecting polyglutamylation such as an anti-polyglutamylation antibody. Examples of the substrates include polypeptides contain a glutamate rich domain such as the polypeptide corresponding to SEQ ID NO: 23, but are not limited to. More preferably, a substrate may be the PELP1 polypeptide or a functional equivalent thereof.

The kit may contain more than one of the aforementioned reagents. Furthermore, the kit may include a solid matrix and reagent for binding a probe against the TTLL4 gene or antibody against the TTLL4 protein, a medium and container for culturing cells, positive and negative control reagents, and a secondary antibody for detecting an antibody against the TTLL4 protein. For example, tissue samples obtained from subject suffering from pancreatic cancer or not may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be included in a container with a label. Suitable containers include bottles, vials, and test tubes. The containers may be formed from a variety of materials, such as glass or plastic.

As an embodiment of the present invention, when the reagent is a probe against the TTLL4 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of TTLL4 mRNA present in the sample. The detection sites may be configured in any suitably detectable shape and are typically in the shape of a bar or dot spanning the width of a test strip.

The kit of the present invention may further include a positive control sample or TTLL4 standard sample. The positive control sample of the present invention may be prepared by collecting TTLL4 positive blood samples and then those TTLL4 level are assayed. Alternatively, purified TTLL4 protein or polynucleotide may be added to TTLL4 free serum to form the positive sample or the TTLL4 standard. In the present invention, purified TTLL4 may be recombinant protein. The TTLL4 level of the positive control sample is, for example more than cut off value.

Screening for an Anti-Cancer Compound:

In the context of the present invention, agents to be identified through the present screening methods may be any compound or composition including several compounds. Furthermore, the test agent exposed to a cell or protein according to the screening methods of the present invention may be a single compound or a combination of compounds. When a combination of compounds is used in the methods, the compounds may be contacted sequentially or simultaneously.

Any test agent, for example, cell extracts, cell culture supernatant, products of fermenting microorganism, extracts from marine organism, plant extracts, purified or crude proteins, peptides, non-peptide compounds, synthetic micromolecular compounds (including nucleic acid constructs, such as antisense RNA, siRNA, Ribozymes, and aptamer etc.) and natural compounds can be used in the screening methods of the present invention. The test agent of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound screened by any of the present screening methods is converted by addition, deletion and/or replacement, is included in the agents obtained by the screening methods of the present invention.

Furthermore, when the screened test agent is a protein, for obtaining a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA is confirmed it's usefulness in preparing the test agent which is a candidate for treating or preventing cancer.

Test agents useful in the screenings described herein can also be antibodies that specifically bind to TTLL4 protein or partial peptides thereof that lack the biological activity of the original proteins in vivo.

Although the construction of test agent libraries is well known in the art, herein below, additional guidance in identifying test agents and construction libraries of such agents for the present screening methods are provided.

(i) Molecular Modeling:

Construction of test agent libraries is facilitated by knowledge of the molecular structure of compounds known to have the properties sought, and/or the molecular structure of TTLL4. One approach to preliminary screening of test agents suitable for further evaluation is computer modeling of the interaction between the test agent and its target.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., J Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor has been identified, combinatorial chemistry techniques can be employed to construct any number of variants based on the chemical structure of the identified putative inhibitor, as detailed below. The resulting library of putative inhibitors, or “test agents” may be screened using the methods of the present invention to identify test agents treating or preventing the pancreatic cancer.

(ii) Combinatorial Chemical Synthesis:

Combinatorial libraries of test agents may be produced as part of a rational drug design program involving knowledge of core structures existing in known inhibitors. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides of six amino acids in length. Such a peptide library could include every 6 amino acid sequence permutation. This type of library is termed a linear combinatorial chemical library.

Preparation of combinatorial chemical libraries is well known to those of skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N. J., Tripos, Inc., St. Louis, Mo., 3D Pharmaceuticals, Exton, Pa., Martek Biosciences, Columbia, Md., etc.).

(iii) Other Candidates:

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Aptamers are macromolecules composed of nucleic acid that bind tightly to a specific molecular target. Tuerk and Gold (Science. 249:505-510 (1990)) discloses SELEX (Systematic Evolution of Ligands by Exponential Enrichment) method for selection of aptamers. In the SELEX method, a large library of nucleic acid molecules {e.g., 10¹⁵ different molecules) can be used for screening.

Screening for an TTLL4 Binding Compound:

In the present invention, over-expression of TTLL4 was detected in pancreatic cancer, in spite of no expression in normal organs (FIG. 1). Therefore, using the TTLL4 genes, proteins encoded by the genes, the present invention provides a method of screening for a compound that binds to TTLL4. Due to the expression of TTLL4 in pancreatic cancer, a compound binds to TTLL4 is expected to suppress the proliferation of cancer cells expressing TTL4, and thus be useful for treating or preventing cancer relating to TTLL4, wherein the cancer is pancreatic cancer. Therefore, the present invention also provides a method for screening a compound that suppresses the proliferation of the cancer cells, and a method for screening a compound for treating or preventing the cancer using the TTLL4 polypeptide. Specially, an embodiment of this screening method includes the steps of:

(a) contacting a test compound with a polypeptide encoded by a polynucleotide of TTLL4;

(b) detecting the binding activity between the polypeptide and the test compound; and

(c) selecting the test compound that binds to the polypeptide.

The method of the present invention will be described in more detail below.

The TTLL4 polypeptide to be used for screening may be a recombinant polypeptide or a protein derived from the nature or a partial peptide thereof. The polypeptide to be contacted with a test compound can be, for example, a purified polypeptide, a soluble protein, a form bound to a carrier or a fusion protein fused with other polypeptides.

As a method of screening for proteins, for example, that bind to the TTLL4 polypeptide using the TTLL4 polypeptide, many methods well known by a person skilled in the art can be used. Such a screening can be conducted by, for example, immunoprecipitation method, specifically, in the following manner. The gene encoding the TTLL4 polypeptide is expressed in host (e.g., animal) cells and so on by inserting the gene to an expression vector for foreign genes, such as pSV2neo, pcDNA I, pcDNA3.1, pCAGGS and pCD8.

The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet. 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by TTLL4 gene can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the TTLL4 polypeptide by the fusion is also reported. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the TTLL4 polypeptide (Experimental Medicine 13: 85-90 (1995)).

In immunoprecipitation, an immune complex is formed by adding these antibodies to cell lysate prepared using an appropriate detergent. The immune complex consists of the TTLL4 polypeptide, a polypeptide including the binding ability with the polypeptide, and an antibody. Immunoprecipitation can be also conducted using antibodies against the TTLL4 polypeptide, besides using antibodies against the above epitopes, which antibodies can be prepared as described above. An immune complex can be precipitated, for example, by Protein A sepharose or Protein G sepharose when the antibody is a mouse IgG antibody. If the polypeptide encoded by TTLL4 gene is prepared as a fusion protein with an epitope, such as GST, an immune complex can be formed in the same manner as in the use of the antibody against the TTLL4 polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the TTLL4 polypeptide is difficult to be detected by a common staining method, such as Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

As a method of screening for proteins binding to the TTLL4 polypeptide using the polypeptide, for example, West-Western blotting analysis (Skolnik et al., Cell 65: 83-90 (1991)) can be used. Specifically, a protein binding to the TTLL4 polypeptide can be obtained by preparing a cDNA library from cultured cells (e.g., Panc-1, MiaPaCa2) expected to express a protein binding to the TTLL4 polypeptide using a phage vector (e.g., ZAP), expressing the protein on LB-agarose, fixing the protein expressed on a filter, reacting the purified and labeled TTLL4 polypeptide with the above filter, and detecting the plaques expressing proteins bound to the TTLL4 polypeptide according to the label. The polypeptide of the invention may be labeled by utilizing the binding between biotin and avidin, or by utilizing an antibody that specifically binds to the TTLL4 polypeptide, or a peptide or polypeptide (for example, GST) that is fused to the TTLL4 polypeptide. Methods using radioisotope or fluorescence and such may be also used.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, the polypeptide of the invention is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A compound binding to the polypeptide encoded by TTLL4 gene can also be screened using affinity chromatography. For example, the polypeptide of the invention may be immobilized on a carrier of an affinity column, and a test compound, containing a protein capable of binding to the polypeptide of the invention, is applied to the column. A test compound herein may be, for example, cell extracts, cell lysates, etc. After loading the test compound, the column is washed, and compounds bound to the polypeptide of the invention can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound compound in the present invention. When such a biosensor is used, the interaction between the polypeptide of the invention and a test compound can be observed real-time as a surface plasmon resonance signal, using only a minute amount of polypeptide and without labeling (for example, BIAcore, Pharmacia). Therefore, it is possible to evaluate the binding between the polypeptide of the invention and a test compound using a biosensor such as BIAcore.

The methods of screening for molecules that bind when the immobilized TTLL4 polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the TTLL4 protein (including agonist and antagonist) are well known to one skilled in the art.

Screening for a Compound Suppressing the Biological Activity of TTLL4:

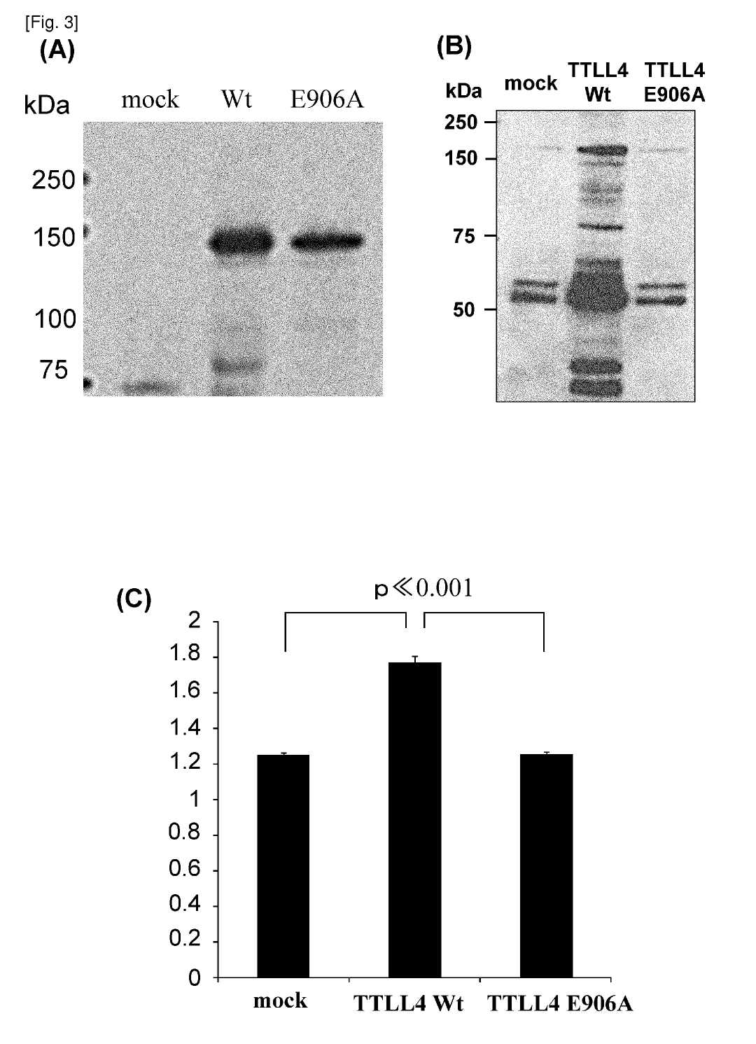

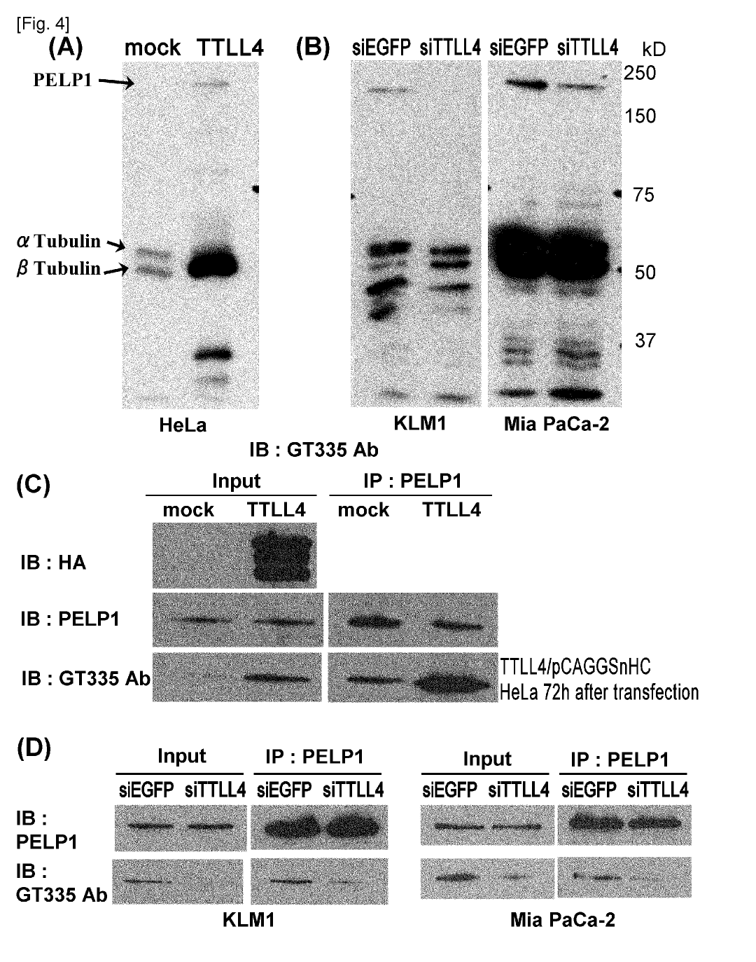

In the present invention the TTLL4 protein have the activity of promoting cell proliferation of pancreatic cancer cells (FIG. 3B) and polyglutamylation activity (FIG. 4B, 5B). Using these biological activities, the present invention provides a method for screening a compound that suppresses the proliferation of cancer cells expressing TTLL4, and a method for screening a compound for treating or preventing cancer relating to TTLL4, wherein the cancer is pancreatic cancer. Thus, the present invention provides a method of screening for a compound for treating or preventing the cancer using the polypeptide encoded by TTLL4 gene including the steps as follows:

(a) contacting a test compound with a polypeptide encoded by a polynucleotide of TTLL4;

(b) detecting the biological activity of the polypeptide of step (a); and

(c) selecting the test compound that suppresses the biological activity of the polypeptide encoded by the polynucleotide of TTLL4 as compared to the biological activity of said polypeptide detected in the absence of the test compound.

According to the present invention, the therapeutic effect of the test compound on suppressing the biological activity (such as the activity to promote cell proliferation or polyglutamylation activity), or a candidate compound for treating or preventing cancer relating to TTLL4 (e.g., pancreatic cancer) may be evaluated. Therefore, the present invention also provides a method of screening for a candidate compound for sup-pressing the cell proliferation, or a candidate compound for treating or preventing cancer relating to TTLL4, using the TTLL4 polypeptide or fragments thereof including the steps as follows:

a) contacting a test compound with the TTLL4 polypeptide or a functional fragment thereof; and

b) detecting the biological activity of the polypeptide or fragment of step (a), and

c) correlating the biological activity of b) with the therapeutic effect of the test agent or compound.

In the present invention, the therapeutic effect may be correlated with the biological activity of the TTLL4 polypeptide or a functional fragment thereof. For example, when the test compound suppresses or inhibits the biological activity of the TTLL4 polypeptide or a functional fragment thereof as compared to a level detected in the absence of the test compound, the test compound may identified or selected as the candidate compound having the therapeutic effect. Alternatively, when the test compound does not suppress or inhibit the biological activity of the TTLL4 polypeptide or a functional fragment thereof as compared to a level detected in the absence of the test compound, the test compound may identified as the agent or compound having no significant therapeutic effect.

The method of the present invention will be described in more detail below.

Any polypeptides can be used for screening so long as they include the biological activity of the TTLL4 protein. Such biological activity includes cell-proliferating activity of the TTLL4 protein. For example, TTLL4 protein can be used and polypeptides functionally equivalent to these proteins can also be used. Such polypeptides may be expressed endogenously or exogenously by cells.

The compound isolated by this screening is a candidate for antagonists of the polypeptide encoded by TTLL4 gene. The term “antagonist” refers to molecules that inhibit the function of the polypeptide by binding thereto. Said term also refers to molecules that reduce or inhibit expression of the gene encoding TTLL4. Moreover, a compound isolated by this screening is a candidate for compounds which inhibit the in vivo interaction of the TTLL4 polypeptide with other molecules (including DNAs and proteins).

When the biological activity to be detected in the present method is cell proliferation, it can be detected, for example, by preparing cells which express the TTLL4 polypeptide, culturing the cells in the presence of a test compound, and determining the speed of cell proliferation, measuring the cell cycle and such, as well as by measuring the colony forming activity, for example, shown in FIG. 2B. The compounds that reduce the speed of proliferation of the cells expressed TTLL4 are selected as candidate compound for treating or preventing pancreatic cancer.

More specifically, the method includes the step of:

(a) contacting a test compound with cells overexpressing TTLL4;

(b) measuring cell-proliferating activity; and

(c) selecting the test compound that reduces the cell-proliferating activity in the comparison with the cell-proliferating activity in the absence of the test compound.

In preferable embodiments, the method of the present invention may further include the steps of:

(d) selecting the test compound that have no effect to the cells no or little expressing TTLL4.

When the biological activity to be detected in the present method is polyglutamylation activity, the polyglutamylation activity can be determined by contacting a polypeptide with a substrate (e.g., PELP1) and a co-factor (e.g., glutamate) under conditions suitable for polyglutamylation of the substrate and detecting the polyglutamylation level of the substrate.

More specifically, the method includes the step of:

[1] contacting a TTLL4 polypeptide or functional equivalent thereof with a substrate to be polyglutamylated and a glutamate as a cofactor in the presence of the test compound under the condition capable of polyglutamylation of the substrate;

[2] detecting the polyglutamylation level of the substrate;

[3] selecting a candidate compound that reduces the polyglutamylation level as compared to a control;

[4] The method of [1], wherein the functional equivalent of TTLL4 polypeptide includes TTL domain corresponding to SEQ ID NO: 22;

[5] The method of [1], wherein the substrate is the polypeptide including glutamate rich domain corresponding to SEQ ID NO: 23; and

[6] The method of [5], wherein the substrate is PELP1.

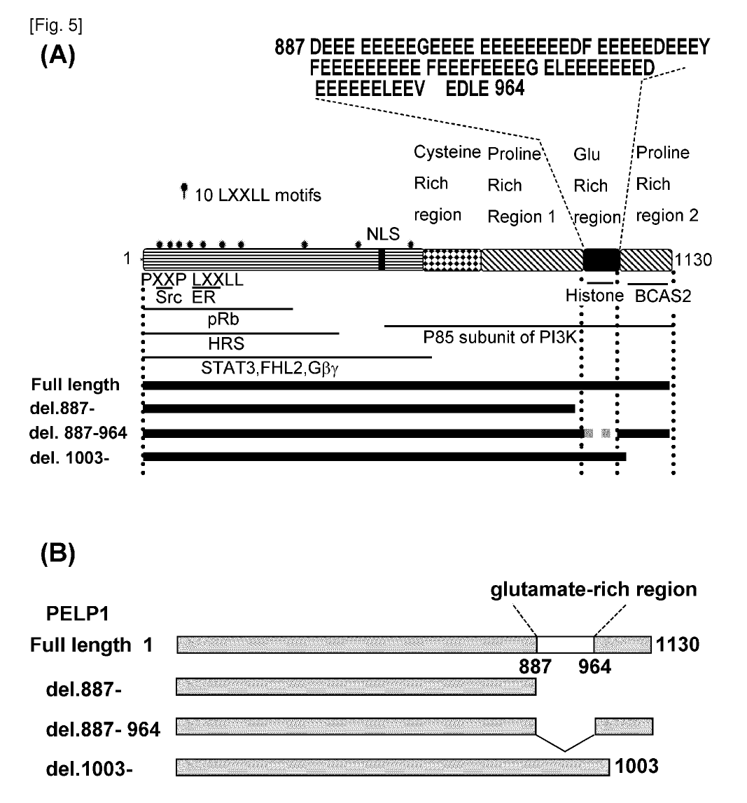

Furthermore, the present method detecting polyglutamylation activity can be performed, for example, by preparing cells which express the TTLL4 polypeptide, culturing the cells in the presence of a test compound, and determining polyglutamylation level of PELP1 by using the antibody specific binding to polyglutamylation region, for example, shown in FIGS. 4 and 5.

More specifically, the method includes the step of:

[1] contacting a test compound with cells expressing TTLL4 and PELP1;

[2] detecting a polyglutamylation level of PELP1; and

[3] selecting the test compound that reduces the polyglutamylation in the comparison with the polyglutamylation in the absence of the test compound.

In the present invention, polyglutamylation activity of a TTLL4 polypeptide or polypeptide includes TTL domain such as SEQ ID NO: 22 can be determined by methods known in the art. For example, the TTLL4 and a substrate can be incubated with a labeled glutamate, under suitable assay conditions. Such the suitable assay condition includes adding the cell lysate. A PELP1 or the polypeptide including glutamate rich domain such as SEQ ID NO: 23 preferably can be used as the substrate. Transfer of the radiolabel to the substrate can be detected, for example, by SDS-PAGE electrophoresis and fluorography. Alternatively, following the reaction the substrate can be separated from the labeled glutamate by filtration, and the amount of radiolabel retained on the filter quantitated by scintillation counting. Other suitable labels that can be attached to glutamate, such as chromogenic and fluorescent labels, and methods of detecting transfer of these labels to the substrate, are known in the art.

Alternatively, polyglutamylation activity of TTLL4 can be determined using an unlabeled glutamate and reagents that selectively recognize polyglutamylation. For example, after incubation of the TTLL4, substrate to be polyglutamylated and glutamate, under the condition capable of polyglutamylation of the substrate, polyglutamylated substrate can be detected by immunological method. Any immunological techniques using an antibody recognizing polyglutamylated substrate can be used for the detection. For example, an antibody against polyglutamylation is available (GT335 antibody: Wolff A et al., 1992 Eur J Cell Biol 59: 425-432). ELISA or Immunoblotting with antibodies recognizing methylated histone can be used for the present invention.

Furthermore, the present invention provides a kit for screening for a compound for treating or cancer relating to TTLL4 or inhibiting TTLL4 expressing cancer cell growth, wherein the compound reduces polyglutamylation activity. Specifically, the kit includes the components of:

(a) a TTLL4 polypeptide or functional equivalent thereof;

(b) a substrate capable of polyglutamylation by the polypeptide of (a);

(c) glutamate; and

(d) a reagent for detecting the polyglutamylation of substrate.

Suitable polypeptide functional equivalent of TTLL4 includes TTL domain corresponding to polypeptide of SEQ ID NO: 22. On the other hand, suitable substrate capable of polyglutamylation includes PELP1 and functional equivalent thereof. The functional equivalent of PELP1 includes glutamate rich domain such as the amino acid sequence of SEQ ID NO: 23. In the present invention, suitable reagent for detecting the polyglutamylation is antibody. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the polyglutamylated protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the polyglutamylation may be included in the kit.

“Suppress the biological activity” as defined herein are preferably at least 10% suppression of the biological activity of TTLL4 in comparison with in absence of the compound, more preferably at least 25%, 50% or 75% suppression and most preferably at least 90% suppression.

Screening for a Compound Altering the Expression of TTLL4:

In the present invention, the decrease of the expression of TTLL4 by siRNA causes inhibiting cancer cell proliferation (FIG. 2C). Therefore, the present invention provides a method of screening for a compound that inhibits the expression of TTLL4. A compound that inhibits the expression of TTLL4 is expected to suppress the proliferation of cancer cells expressing TTLL4, and thus is useful for treating or preventing cancer relating to TTLL4. Therefore, the present invention also provides a method for screening a compound that suppresses the proliferation of the cancer cells, and a method for screening a compound for treating or preventing the cancer. In the context of the present invention, such screening may include, for example, the following steps:

(a) contacting a candidate compound with a cell expressing TTLL4; and

(b) selecting the candidate compound that reduces the expression level of TTLL4 as compared to a control.

According to the present invention, the therapeutic effect of the test compound on suppressing the expression level of TTLL4, or a candidate compound for treating or preventing cancer relating to TTLL4 (e.g., pancreatic cancer) may be evaluated. Therefore, the present invention also provides a method of screening for a candidate compound for suppressing the expression level of TTLL4, or a candidate compound for treating or preventing cancer relating to TTLL4, including the steps as follows:

a) contacting a candidate compound with a cell expressing TTLL4; and

b) evaluating the expression level of TTLL4, and

c) correlating the expression level of TTLL4 of b) with the therapeutic effect of the test compound.

In the present invention, the therapeutic effect may be correlated with the expression level of TTLL4. For example, when the test compound reduces the expression level of TTLL4 as compared to a level detected in the absence of the test compound, the test compound may identified or selected as the candidate compound having the therapeutic effect. Alternatively, when the test compound does not reduce the expression level of TTLL4 as compared to a level detected in the absence of the test compound, the test compound may identified as the agent or compound having no significant therapeutic effect.

The method of the present invention will be described in more detail below.

Cells expressing the TTLL4 include, for example, cell lines established from pancreatic cancer; such cells can be used for the above screening of the present invention (e.g., Panc-1, MiaPaCa2). The expression level can be estimated by methods well known to one skilled in the art, for example, RT-PCR, Northern blot assay, Western blot assay, immunostaining and flow cytometry analysis. “Reduce the expression level” as defined herein are preferably at least 10% reduction of expression level of TTLL4 in comparison to the expression level in absence of the compound, more preferably at least 25%, 50% or 75% reduced level and most preferably at least 95% reduced level. The compound herein includes chemical compounds, double-strand nucleotides, and so on. The preparation of the double-strand nucleotides is in aforementioned description. In the method of screening, a compound that reduces the expression level of TTLL4 can be selected as candidate compounds to be used for the treatment or prevention of pancreatic cancer.

Alternatively, the screening method of the present invention may include the following steps:

(a) contacting a candidate compound with a cell into which a vector, including the transcriptional regulatory region of TTLL4 and a reporter gene that is expressed under the control of the transcriptional regulatory region, has been introduced;

(b) measuring the expression or activity of said reporter gene; and

(c) selecting the candidate compound that reduces the expression or activity of said reporter gene.

Suitable reporter genes and host cells are well known in the art. For example, reporter genes are luciferase, green florescence protein (GFP), Discosoma sp. Red Fluorescent Protein (DsRed), Chrolamphenicol Acetyltransferase (CAT), lacZ and beta-glucuronidase (GUS), and host cell is COS7, HEK293, HeLa and so on. The reporter construct required for the screening can be prepared by connecting reporter gene sequence to the transcriptional regulatory region of TTLL4. The transcriptional regulatory region of TTLL4 herein is the region from transcription start site to at least 500 bp upstream, preferably 1000 bp, more preferably 5000 or 10000 bp upstream. A nucleotide segment containing the transcriptional regulatory region can be isolated from a genome library or can be propagated by PCR. The reporter construct required for the screening can be prepared by connecting reporter gene sequence to the transcriptional regulatory region of any one of these genes. Methods for identifying a transcriptional regulatory region, and also assay protocol are well known (Molecular Cloning third edition chapter 17, 2001, Cold Springs Harbor Laboratory Press).

The vector containing the reporter construct is introduced into host cells and the expression or activity of the reporter gene is detected by methods well known in the art (e.g., using luminometer, absorption spectrometer, flow cytometer and so on). “Reduces the expression or activity” as defined herein are preferably at least 10% reduction of the expression or activity of the reporter gene in comparison with in absence of the compound, more preferably at least 25%, 50% or 75% reduction and most preferably at least 95% reduction.

Screening for a Compound Decreasing the Binding Between TTLL4 and PELP1:

In the present invention, the interaction between TTLL4 and PELP1 is shown by immunoprecipitation (FIG. 4D). Additionally, TTLL4 polyglutamylates PELP1 (FIG. 4D). Therefore, the present invention provides a method of screening for a compound that inhibits the binding between TTLL4 and PELP1. A compound that inhibits the binding between TTLL4 and PELP1 is expected to suppress the proliferation of cancer cells expressing TTLL4, and thus is useful for treating or preventing cancer relating to TTLL4. Therefore, the present invention also provides a method for screening a compound that inhibits the binding between TTLL4 and PELP1 and suppresses the proliferation of the cancer cells, and a method for screening a compound for treating or preventing the cancer.

More specifically, the method includes the steps of:

(a) contacting TTLL4 polypeptide or functional equivalent thereof with PELP1 or functional equivalent thereof, in the presence of a test compound;

(b) detecting the binding between the polypeptides; and

(c) selecting the test compound that inhibits the binding between the polypeptides.

Herein, the phrase “functional equivalent of TTLL4 polypeptide” as used herein refers to the polypeptide which includes amino acid sequence of PELP1 binding domain. Similarly, the term “functional equivalent of PELP1 polypeptide” refers to the polypeptide which includes amino acid sequence of TTLL4 binding domain.

The method of the present invention is described in further detail below.

As a method of screening for compounds that inhibit binding between TTLL4 and PELP1 many methods well known by one skilled in the art can be used. Such a screening can be carried out as an in vitro assay system. More specifically, first, TTLL4 polypeptide is bound to a support, and PELP1 polypeptide is added together with a test compound thereto. Next, the mixture is incubated, washed and PELP1 polypeptide bound to the support is detected and/or measured. Promising candidate compound can reduce the amount of detecting PELP1 polypeptide. On the contrary, PELP1 polypeptide may be bound to a support and TTLL4 polypeptide may be added. Here, TTLL4 and PELP1 can be prepared not only as a natural protein but also as a recombinant protein prepared by the gene recombination technique. The natural protein can be prepared, for example, by affinity chromatography. On the other hand, the recombinant protein may be prepared by culturing cells transformed with DNA encoding TTLL4 or PELP1 to express the protein therein and then recovering it.

Examples of supports that may be used for binding proteins include insoluble polysaccharides, such as agarose, cellulose and dextran; and synthetic resins, such as polyacrylamide, polystyrene and silicon; preferably commercial available beads and plates (e.g., multi-well plates, biosensor chip, etc.) prepared from the above materials may be used. When using beads, they may be filled into a column. Alternatively, the use of magnetic beads of also known in the art, and enables to readily isolate proteins bound on the beads via magnetism.

The binding of a protein to a support may be conducted according to routine methods, such as chemical bonding and physical adsorption. Alternatively, a protein may be bound to a support via antibodies specifically recognizing the protein. Moreover, binding of a protein to a support can be also conducted by means of avidin and biotin. The binding between proteins is carried out in buffer, for example, but are not limited to, phosphate buffer and Tris buffer, as long as the buffer does not inhibit binding between the proteins.

In the present invention, a biosensor using the surface plasmon resonance phenomenon may be used as a mean for detecting or quantifying the bound protein. When such a biosensor is used, the interaction between the proteins can be observed real-time as a surface plasmon resonance signal, using only a minute amount of polypeptide and without labeling (for example, BIAcore, Pharmacia). Therefore, it is possible to evaluate binding between TTLL4 and PELP1 using a biosensor such as BIAcore.

Alternatively, TTLL4 or PELP1 may be labeled, and the label of the polypeptide may be used to detect or measure the binding activity. Specifically, after pre-labeling one of the polypeptide, the labeled polypeptide is contacted with the other polypeptide in the presence of a test compound, and then bound polypeptide are detected or measured according to the label after washing. Labeling substances such as radioisotope (e.g., ³H, ¹⁴C, ³²P, ³³P, ³⁵S, ¹²⁵I, ¹³¹I), enzymes (e.g., alkaline phosphatase, horseradish peroxidase, beta-galactosidase, b-glucosidase), fluorescent substances (e.g., fluorescein isothiocyanate (FITC), rhodamine) and biotin/avidin, may be used for the labeling of a protein in the present method. When the protein is labeled with radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Furthermore, binding between TTLL4 and PELP1 can be also detected or measured using antibodies to TTLL4 or PELP1. For example, after contacting TTLL4 polypeptide immobilized on a support with a test compound and PELP1 polypeptide mixture is incubated and washed, and detection or measurement can be conducted using an antibody against PELP1 polypeptide.

Alternatively, PELP1 polypeptide may be immobilized on a support, and an antibody against TTLL4 may be used as the antibody. In case of using an antibody in the present screening, the antibody is preferably labeled with one of the labeling substances mentioned above, and detected or measured based on the labeling substance. Alternatively, the antibody against TTLL4 or PELP1 polypeptide may be used as a primary antibody to be detected with a secondary antibody that is labeled with a labeling substance. Furthermore, the antibody bound to the protein in the screening of the present invention may be detected or measured using protein G or protein A column.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, for example, TTLL4 polypeptide is fused to the SRF-binding region or GAL4-binding region and expressed in yeast cells. PELP1 polypeptide that binds to TTLL4 polypeptide that binds to PELP1 polypeptide is fused to the VP16 or GAL4 transcriptional activation region and also expressed in the yeast cells in the existence of a test compound. Alternatively, TTLL4 polypeptide may be fused to the SRF-binding region or GAL4-binding region, and PELP1 polypeptide to the VP16 or GAL4 transcriptional activation region. The binding of the two activates a reporter gene, making positive clones detectable. As a reporter gene, for example, Ade2 gene, lacZ gene, CAT gene, luciferase gene and such can be used besides HIS3 gene.

Screening for a Compound Modulating the Binding Between PELP1 and LAS1L or SENP3:

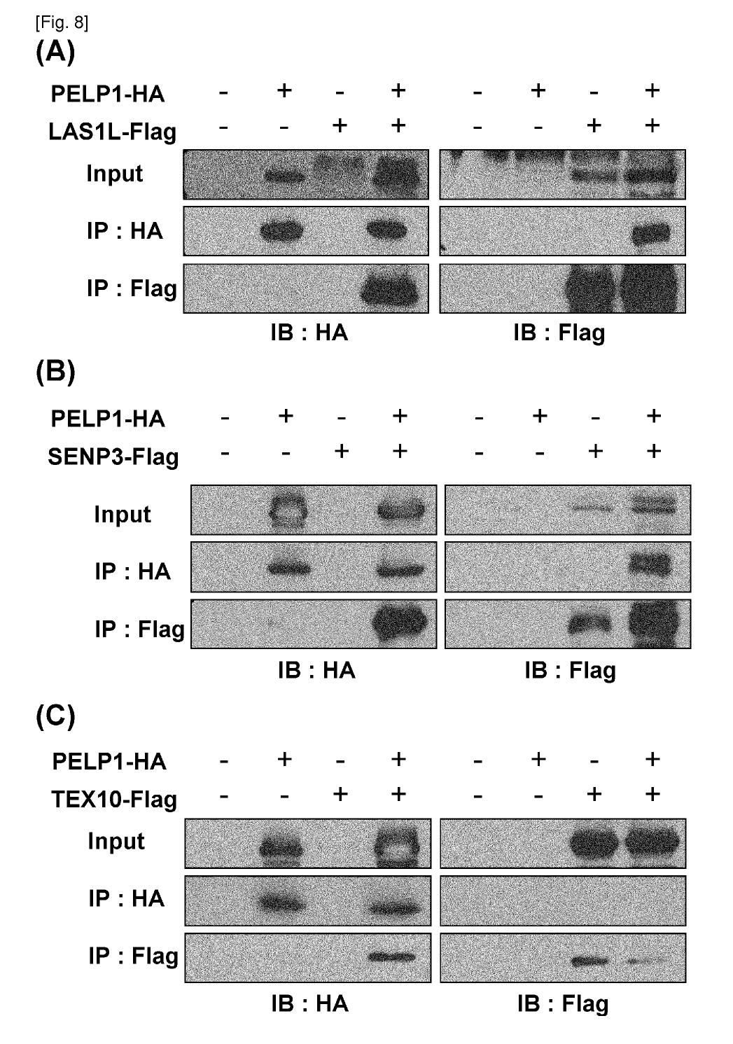

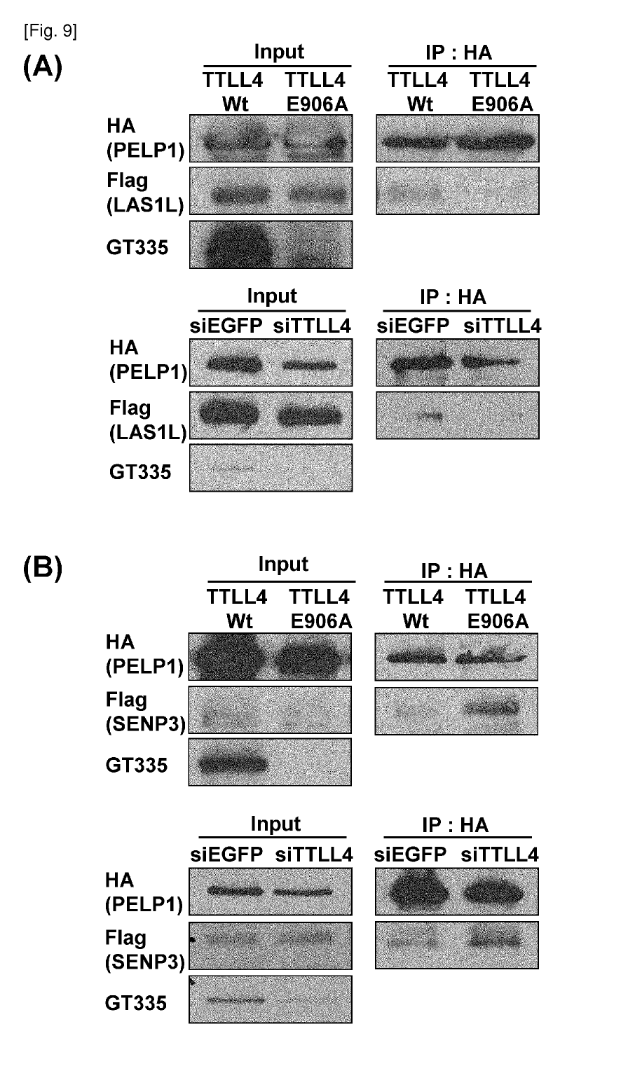

In the present invention, the interaction between PELP1 and LAS1L or SENP3 is shown by immunoprecipitation (FIG. 8). Additionally, the over-expression of TTLL4 elevated an amount of LAS1L binding with PELP1, and the suppression of TTLL4 expression reduced an amount of LAS1L binding with PELP1, as compared to the controls (FIG. 9A). On the other hand, the over-expression of TTLL4 reduced an amount of SENP3 binding with PELP1, and the suppression of TTLL4 expression elevated an amount of SENP3 binding with PELP1, as comparing to the controls (FIG. 9B). Therefore, the binding activity between PELP1 and, LAS1L or SENP3 may be used as a index of the biological activity of TTLL4. A compound that modulates the binding between PELP1 and, LAS1L or SENP3 is expected to suppress the proliferation of cancer cells expressing TTLL4, and thus is useful for treating or preventing cancer relating to TTLL4. Therefore, the present invention also provides a method for screening a compound that inhibits the binding between PELP1 and, LAS1L or SENP3 (as well as TTLL4 and PELP1) and suppresses the proliferation of the cancer cells, and a method for screening a compound for treating or preventing the cancer.

More specifically, the method includes the steps of:

(a) contacting PELP1 polypeptide or functional equivalent thereof with LAS1L or functional equivalent thereof, in the presence of TTLL4 polypeptide or a functional equivalent thereof, and a test compound;

(b) detecting the binding between the PELP1 polypeptide or functional equivalent thereof and LAS1L or functional equivalent; and

(c) selecting the test compound that inhibits the binding between the polypeptides.

Alternatively, the method include the steps of:

(a) contacting PELP1 polypeptide or functional equivalent thereof with SENP3 or functional equivalent thereof, in the presence of TTLL4 polypeptide or a functional equivalent thereof, and a test compound;

(b) detecting the binding between the PELP1 polypeptide or functional equivalent thereof and SENP3 or functional equivalent; and

(c) selecting the test compound that enhances the binding between the polypeptides.

The examples of functional equivalent of TTLL4 polypeptide include polypeptides having an amino acid sequence of a TTL domain corresponding to SEQ ID NO:22, and the examples of functional equivalent of PELP1 polypeptide include polypeptides having an amino acid sequence of a glutamate rich domain corresponding to SEQ ID NO:23.

The method of the present invention is described in further detail below.

As a method of screening for compounds that modulating binding between PELP1 and LAS1L or SENP3, many methods well known by one skilled in the art can be used. Such a screening can be carried out as an in vitro assay system. More specifically, first, PELP1 polypeptide is bound to a support, and the LAS1L or SENP3 polypeptide is added together with TTLL4 polypeptide and a test compound, thereto. Preferably, a glutamate may be further added together thereto. Next, the mixture is incubated, washed and the LAS1L or SENP3 polypeptide bound to the support is detected and/or measured. When the LAS1L polypeptide is added, promising candidate compound may reduce the amount of detecting the LSA1L polypeptide. On the other hand, when the SENP3 polypeptide is added, promising candidate compound may increase the amount of detecting the NENP3 polypeptide. Alternatively, the LAS1L or SENP3 polypeptide may be bound to a support and the PELP1 polypeptide may be added thereto.

The TTLL4, PELP1, LAS1L and SENP3 polypeptide may be prepared not only as a natural protein but also as a recombinant protein prepared by the gene recombination technique based on their nucleotide sequences. The natural protein can be prepared, for example, by affinity chromatography. On the other hand, the recombinant protein may be prepared by culturing cells transformed with DNA encoding TTLL4, PELP1, LAS1L or SENP3 to express the protein therein and then recovering it.

Examples of supports that may be used for binding proteins include insoluble polysaccharides, such as agarose, cellulose and dextran; and synthetic resins, such as polyacrylamide, polystyrene and silicon; preferably commercial available beads and plates (e.g., multi-well plates, biosensor chip, etc.) prepared from the above materials may be used. When using beads, they may be filled into a column. Alternatively, the use of magnetic beads of also known in the art, and enables to readily isolate proteins bound on the beads via magnetism.

The binding of a protein to a support may be conducted according to routine methods, such as chemical bonding and physical adsorption. Alternatively, a protein may be bound to a support via antibodies specifically recognizing the protein. Moreover, binding of a protein to a support can be also conducted by means of avidin and biotin. The binding between proteins is carried out in buffer, for example, but are not limited to, phosphate buffer and Tris buffer, as long as the buffer does not inhibit binding between the proteins.

In the present invention, a biosensor using the surface plasmon resonance phenomenon may be used as a mean for detecting or quantifying the bound protein. When such a biosensor is used, the interaction between the proteins can be observed real-time as a surface plasmon resonance signal, using only a minute amount of polypeptide and without labeling (for example, BIAcore, Pharmacia). Therefore, it is possible to evaluate binding between PELP1 and LAS1L or SENP3 using a biosensor such as BIAcore.

Alternatively, PELP1 or, LAS1L or SENP3 may be labeled, and the label of the polypeptide may be used to detect or measure the binding activity. Specifically, after pre-labeling one of the polypeptide, the labeled polypeptide is contacted with the other polypeptide in the presence of TTLL4 and a test compound, and then bound polypeptide are detected or measured according to the label after washing. Labeling substances such as radioisotope (e.g., ³H, ¹⁴C, ³²P, ³³P, ³⁵S, ¹²⁵I, ¹³¹I), enzymes (e.g., alkaline phosphatase, horseradish peroxidase, beta-galactosidase, b-glucosidase), fluorescent substances (e.g., fluorescein isothiocyanate (FITC), rhodamine) and biotin/avidin, may be used for the labeling of a protein in the present method. When the protein is labeled with radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Furthermore, binding between the polypeptides can be also detected or measured using antibodies to PELP1 or, LAS1L or SENP3. For example, the LAS1L or SENP3 polypeptide, which binds to the PELP1 polypeptide immobilized on a support, may be detected by an anti-LAS1L antibody or an anti-SENP3 antibody respectively. On the contrary, the PELP1 polypeptide, which binds to the LAS1L or SENP3 polypeptide immobilized on a support, may be detected by an anti-PELP1 antibody. In case of using an antibody in the present screening, the antibody is preferably labeled with one of the labeling substances mentioned above, and detected or measured based on the labeling substance. Alternatively, the antibody against the polypeptides may be used as a primary antibody to be detected with a secondary antibody that is labeled with a labeling substance. Furthermore, the antibody bound to the protein in the screening of the present invention may be detected or measured using protein G or protein A column.

According to the present invention, the therapeutic effect of a test compound on inhibiting (i) the binding between TTLL4 and PELP1, or (ii) the binding between PELP1 and, LAS1L or SENP3, or a candidate compound for treating or preventing cancer (such as pancreatic cancer) may be evaluated. Therefore, the present invention also provides a method for screening a candidate compound that suppresses the binding of (i) or (ii) above, and a method for screening a candidate compound for treating or preventing cancer (such as pancreatic cancer).

More specifically, the method includes the steps of:

(a1) contacting TTLL4 polypeptide or functional equivalent thereof with PELP1 or functional equivalent thereof, in the presence of a test compound; or

(a2) contacting PELP1 polypeptide or functional equivalent thereof with LAS1L or SENP3 or functional equivalent thereof, in the presence of TTLL4 polypeptide or a functional equivalent thereof, and a test compound;

and

(b) detecting the level of binding between (i) TTLL4 and PELP1, or (ii) PELP1 and, LAS1L or SENP3; and

(c) comparing the binding level of (b) with that detected in the absence of the test compound; and

(d) correlating the binding level of (b) with the therapeutic effect of the test compound.

In the present invention, the therapeutic effect may be correlated with the binding between (ii) TTLL4 and PELP1, or (ii) PELP1 and, LAS1L or SENP3. For example, when the test compound inhibits the binding between the above-mentioned polypeptides as compared to a level detected in the absence of the test compound, the test compound may identified or selected as the candidate compound having the therapeutic effect. Alternatively, when the test compound does not inhibit the binding between the above-mentioned polypeptides as compared to a level detected in the absence of the test compound, the test compound may identified as the agent or compound having no significant therapeutic effect.

By screening for candidate compounds that (i) bind to the TTLL4 polypeptide; (ii) suppress the biological activity of the TTLL4 polypeptide; (iii) reduce the expression level of TTLL4; (iv) inhibit the binding between TTLL4 and PELP1; (v) inhibit the polyglutamylation of a substrate by TTLL4, (v) modulate the binding between PELP1 and LAS1L or SENP3, candidate compounds that have the potential to treat or prevent cancers (e.g., pancreatic cancer) can be identified. Potential of these candidate compounds to treat or prevent cancers may be evaluated by second and/or further screening to identify therapeutic agent for cancers. For example, when a compound that binds to the TTLL4 polypeptide inhibits above-described activities of cancer, it may be concluded that such a compound has the TTLL4-specific therapeutic effect.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be further described in the following examples, which do not limit the scope of the invention described in the claims.

### EXAMPLES

### I. Materials and Methods

1. Cell Lines

PDAC cell lines KLM-1, SUIT-2, KP-1N, PK-1, PK-45P and PK-59 were provided from Cell Resource Center for Biomedical Research, Tohoku University (Sendai, Japan). PDAC cell lines MIA-PaCa-2 and Panc-1, and COS-7 and HeLa were purchased from the American Type Culture Collection (ATCC, Rockville, Md.). The cell lines, KLM-1, SUIT-2, PK-1, PK-45P, PK-59 and Panc-1, were grown in RPMI1640 (Sigma-Aldorich, St. Louis, Mo.), and MIA-PaCa-2, COS-7 and HeLa in Dulbecco's Modified Eagle's Medium (Sigma-Aldrich), all with 10% fetal bovine serum and 1% antibiotic/antimycotic solution (Sigma-Aldrich). Cells were maintained at 37 degrees C. in an atmosphere of humidified air with 5% CO₂. Frozen and paraffin-embedded PDAC tissues were obtained from surgical specimens that were resected in Osaka Medical Center for Cancer and Cardiovascular Diseases under the appropriate informed consent, and this study using these clinical samples were approved by the institutional review board of Institute of Medical Science, The University of Tokyo, and Osaka Medical Center for Cancer and Cardiovascular Diseases.

2. Semi-Quantitative RT-PCR

Purification of PDAC cells and normal pancreatic ductal epithelial cells from frozen PDAC tissues was described previously (Nakamura T et al., Oncogene 2004, 23: 2385-400). RNAs from the purified PDAC cells and normal pancreatic ductal epithelial cells were subjected to two rounds of RNA amplification using T7-based in vitro transcription (Epicentre Technologies, Madison, Wis., USA). Total RNAs from human PDAC cell lines were extracted using Trizol reagent (Invitrogen) according to the manufacturer's recommendations. Extracted RNAs were treated with DNase I (Roche Diagnostic, Mannheim, Germany) and reversely-transcribed to single-stranded cDNAs using oligo (dT) primer with Superscript II reverse transcriptase (Invitrogen). Appropriate dilutions of each single-stranded cDNA were prepared for subsequent PCR amplification by monitoring tubulin beta (TUBA) as a quantitative control. The sets of primer sequences were 5′-AAGGATTATGAGGAGGTTGGTGT-3′(SEQ ID NO: 3) and 5′-CTTGGGTCTGTAACAAAGCATTC-3′ (SEQ ID NO: 4) for TUBA, 5′-GTGAGGTCAGCCTACTACTCTCTGAAG-3′(SEQ ID NO: 1) and 5′-CAGGAGGGAGTCACCGATTG-3′(SEQ ID NO: 2) for TTLL4. All reactions involved initial denaturation at 94 degrees C. for 2 min followed by 23 cycles (for TUBA), 29 cycles (for TTLL4) at 94 degrees C. for 30 s, 58 degrees C. for 30 s, and 72 degrees C. for 1 min, on a GeneAmp PCR system 9700 (PE Applied Biosystems, Foster, Calif., USA).

3. Northern Blotting Analysis

One micro-g poly A+ RNAs from eight PDAC cell lines using RNeasy Mini kit (QIAGEN, Valencia, Calif.). Their mRNA was purified with mRNA Purification Kit (GE Healthcare, Piscataway, N.J.), according to the manufacturer's protocols. One micro-g of each mRNA from pancreatic cancer cell lines (KLM-1, PK-59, PK-45P, MIAPaCa-2, Panc-1, PK-1, SUIT-2 and KP-1N) and seven adult normal tissues (heart, lung, liver, kidney, brain, testis and pancreas, from BD Bioscience, Palo Alto, Calif.) were separate on 1% denaturing agarose gels and transferred onto a nylon membrane. This northern-blot membrane and human MTN blot membrane (Multiple Tissue Northern blot, BD Bioscience) were hybridized for 16 hours with ³²P-labeled GABRP probe, which was labeled using Mega Label kit (GE Healthcare, Piscataway, N.J.). Probe cDNA of TTLL4 was prepared as a 474-bp PCR product by using primers 5′-ATCAAAGGCCAGATGATTCG-3′ (SEQ ID NO: 5) and 5′-GACAACTCCCAT-GTGGAACC-3′ (SEQ ID NO: 6). Pre-hybridization, hybridization, and washing were performed according to the manufacturer's instruction. The blots were autoradiographed at −80 degrees C. for 10 days.

4. Small Interfering RNA (siRNA)-Expressing Vectors Specific to TTLL4

To knock down endogenous TTLL4 expression in PDAC cells, psiU6BX3.0 vector was used for expression of short hairpin RNA against a target gene as described previously (Taniuchi K et al., 2005 Cancer Res 65 : 105-12). The U6 promoter was cloned upstream of the gene-specific sequence (19-nt sequence from the target transcript, separated from the reverse complement of the same sequence by a short spacer, TTCAAGAGA), with five thymidines as a termination signal and a neo cassette for selection by Geneticin (Sigma-Aldrich). The target sequences for TTLL4 were 5′-GAAGCAAGTGGAGacactg-3′ (si-#466) (SEQ ID NO: 7), 5′-GAGCCTTGGCAATaagttc-3′ (si-#2692) (SEQ ID NO: 8), 5′-CATTGTCAAGCAGaccatt-3′ (si-#2146) (SEQ ID NO: 9), and 5′-GAAGCAGCACGACTTCTTC-3′ (siEGFP) (SEQ ID NO: 10) as a negative control. Human PDAC cell lines, Panc-1 or MiaPaca2, were seeded on 10-cm dishes, and transfected with these siRNA-expression vectors using FuGENE6 (Roche) according to manufacturer's instruction, followed by 1000 micro-g/ml (for Panc-1) or 800 micro-g/ml (for MIAPaCa2) Geneticin (GIBCO) selection. The cells from 10-cm dishes were harvested 7 days later to analyze the knockdown effect on TTLL4 by RT-PCR using the above primers. After cultured in appropriate medium containing Geneticin for 12 days, the cells were fixed with 100% methanol, stained with 0.1% of crystal violet-H₂O for colony formation assay. In MTT assay, cell viability was measured using Cell-counting kit-8 (DOJINDO, Kumamoto, Japan) at 6 days after the transfection. Absorbance was measured at 490 nm, and at 630 nm as reference, with a Microplate Reader 550 (Bio-Rad, Hercules, Calif.).

5. Expression Constructs for TTLL4 and PELP1

For full-length cDNA encoding human TTLL4, the PCR product amplified by forward primer: 5′-AGAATTCTGTGGGCCCCTCATGGCCTCAGCAGGA-3′(SEQ ID NO: 11) and reverse primer: 5′-TCGAGCGGCCGCGATGGGCTCACAGCCAGGAGGGA-3′ (SEQ ID NO: 12) and it was cloned into the EcoR1 and Not1 sites of pCAGGS vector and sequenced. Enzyme-dead TTLL4 mutant (E906A), which was reported to lose its enzyme activity of polyglutamylation (van Dijk J et al., 2007 Mol Cell 26: 437-48), was generated by using QuikChange XL Site-Directed Mutagenesis kit (STRATAGENE) and primers 5′-GCCCTGGGTCCTGGCAGTCAACATTTCCCC-3′(SEQ ID NO: 13). Full-length His-tagged PELP1 expression vector was kindly provided from Dr. Ratna K. Vadlamudi (Vadlamudi R K et al., 2005 Cancer Res 65: 7724-7732). For the mutant del 887- of PELP1, PCR product was amplified by forward primer: 5′-TGAATTCATGGCGGCAGCCGTTCTGAGT-3′ (SEQ ID NO: 14) and reverse primer: 5′-TCGAGCGGCCGCGAACTGCTGTTGATATTAATAA-3 (SEQ ID NO: 15), and it was cloned into the EcoR1 and Not1 sites of pCAGGS vector and sequenced. For the mutant del 887-964 of PELP1, the insert of the mutant del 887- and the PCR product amplified by forward primer: 5′-TTTGGCACAGCAGGAGGGGA-3′(SEQ ID NO: 16) and the reverse primer: 5′-TCGAGCGGCCGCGAGGAGTCAGGCTCTGT-3′ (SEQ ID NO: 17) were ligated, which was cloned into the EcoR1 and Not1 sites of pCAGGS vector and sequenced. For the mutant del 1003- of PELP1, PCR product was amplified by forward primer: 5′-TGAATTCATGGCGGCAGCCGTTCTGAGT-3′ (SEQ ID NO: 14) and reverse primer: 5′-TCGAGCGGCCGCAACTCCAGGTCTTCCACCTC-3′ (SEQ ID NO: 24) and it was cloned into the EcoR1 and Not1 sites of pCAGGS vector and sequenced.

6. Cell Growth Assay

COS7 cells were seeded on 10 cm dishes and transfected with 8 ug of wild-type TTLL4, mutant TTLL4 (E906A), or an empty vector. 48 hours after the transfection, the cell viability was evaluated by MTT assay described above.

7. Western Blot Analysis and Immunoprecipitation

COS-7 or Hela cells were transfected with TTLL4 expression vector and/or PELP1 expression vector, and MIAPaCa-2 cells were transfected with si196 duplex (5′-GAAGCAAGUGGAGACACUG-3′: for the target sequence of SEQ ID NO: 7) to down regulate endogenous TTLL4, or siEGFP duplex (5′-GAAGCAGCACGACUUCUUC-3′: for the target sequence of SEQ ID NO: 10) as a negative control. They were collected 48 hours after transfection The cell was lysed with 0.4% NP-40 lysis buffer [0.4% NP-40, 150 mM NaCl, 50 mM Tris-HCl, Protease Inhibitor Cocktail Set III (Calbiochem, San Diego, Calif., USA), pH 8.0] and the cell lysate were subject to SDS-PAGE and western blot analysis using anti-HA antibody (3F10, Roche, Basel, Switzerland), anti-FLAG M2 antibody (SIGMA), anti-PELP1 antibody (A300-180A, BETHYL), or anti-polyglutamylation GT335 antibody (Wolff A et al., 1992 Eur J Cell Biol 59: 425-432). For immunoprecipitation, cells were lysed by lysis buffer (50 mM Tris-HCl [pH7.0], 250 mM sucrose, 1 mM DTT, 10 mM EDTA, 1 mM EGTA, 5 mM MgCl₂), and the cell lysate was immunoprecipitated by anti-PELP1 antibody (A300-876A, BETHYL), and these immunoprecipitated products were subject with western blot analysis described above.

8. Histone H3 Interaction and Modification

PK1 or HeLa cell were lysed with 0.4% NP-40 lysis buffer and the cell lysate was immunoprecipitated by anti-PELP1 antibody (A300-876A, Bethyl), and these immuno-precipitated complexes were subject with Western blot analysis by using Histone H3 antibody (ab1791, abcam, Cambridge, UK). Acetylation and methylation of Histone H3 were evaluated by Western blot analysis by using anti-acetyl-Histone H3 Ab (06-599, Millipore, Bedford, Mass., USA).

9. Immunoprecipitation and Mass-Spectrometric Analysis (LC-MS/MS) for PELP1-Interacting Proteins

PK-1 cells were collected and lysed with homogenate buffer (0.25M sucrose, 10 mM Tris-HCl, 1 mM EDTA, Protease Inhibitor Cocktail Set III, pH 7.4) and centrifuged at 2,300 g for 10 min at 4 degrees C. to separate nuclear fraction. The pellet was washed twice with PBS, lysed with 0.4% NP-40 lysis buffer, and incubated on ice for 30 min. The lysate was incubated with 5 micro-g of anti-PELP1 antibody (Bethyl) or rabbit IgG (Santa Cruz Biotechnology, Santa Cruz, Calif.) at 4 degrees C. for 1.5 hours. Immunocomplexes were incubated with 50 micro-1 of protein G Sepharose (Invitrogen) for 1 hour and washed with lysis buffer. Co-precipitated proteins were separated in 7.5% SDS-PAGE gel and stained with silver-staining kit (Invitrogen). Bands that specifically appeared in the precipitates with PELP1 antibody, but not in those rabbit IgG, were excised, and analyzed them by LC/MS/MS analysis. The excised bands were reduced in 10 mM tris(2-carboxyethyl)phosphine (Sigma) with 50 mM ammonium bicarbonate (Sigma) for 30 min at 37 degrees C. and alkylated in 50 mM iodoacetamide (Sigma) with 50 mM ammonium bicarbonate for 45 min in the dark at 25 degrees C. Porcine trypsin (Promega, San Luis Obispo, Calif.) was added for a final enzyme to protein in ratio of 1:20. The digestion was conducted at 37 degrees C. for 16 hours. The resulting peptide mixture was separated on a 100 micro-m×150 mm HiQ-Sil C18W-3 column (KYA Technologies, Tokyo, Japan) using 30 min linear gradient from 5.4 to 29.2% acetonitrile in 0.1% trifluoroacetic acid (TFA) with total flow of 300 nl/min. The eluting peptides were automatically mixed with matrix solution (4 mg/ml alpha-cyano-4-hydroxy-cinnamic acid (SIGMA), 0.08 mg/ml ammonium citrate in 70% acetonitrile, 0.1% TFA) and spotted onto MALDI target plates by MaP (KYA Technologies). Mass spectrometric analysis was performed on 4800 Plus MALDI/TOF/TOF Analyzer (Applied Biosystems/MDS Sciex). MS/MS peak list was generated by the Protein Pilot version 2.0.1 software (Applied Biosystems/MDS Sciex) and exported to a local MASCOT search engine version 2.2.03 (Matrix Science) for protein data base search.

10. PELP1 Interaction with SENP3 and LAS1L

To confirm the interaction between PELP1 and SENP3 or LAS1L proteins, PELP1-HA expression vector and SENP3-Flag or LAS1L-Flag expression vector were co-transfected into COS-7 cells. For full-length cDNA encoding human SENP3, the PCR product amplified by forward primer: 5′-ATTCGCGGCCGCATGAAAGA-GACTATACAA-3′ (SEQ ID: 25) and reverse primer: 5′-CCGCTCGAGCACAGT-GAGTTTGCAGTGA-3′ (SEQ ID: 26) and they were cloned into the Not1 and Xho1 sites of pCAGGSn3FC vector and sequenced. For full-length cDNA encoding human LAS1L, the PCR product amplified by forward primer: 5′-TGAATTCAT-GTCGTGGGAATCCG-3′ (SEQ ID: 27) and reverse primer: 5′-TCGAGCGGCCGC-GAGAAGAGCTGCAGGCCAGTTT-3′ (SEQ ID: 28) and it was cloned into the EcoR1 and Not1 sites of pCAGGSn3FC vector and sequenced. The transfected cells were lysed as described above and immunoprecipitated with rat anti-HA antibody (Roche, clone3F10) or Flag M2 agarose affinity gel (Sigma). To examine interaction of PELP1-HA and SENP3-Flag or LAS1L-Flag proteins, these immune complexes were analyzed by western blotting with rabbit anti-FLAG or anti-HA antibodies.

### II. Results

1. Over-Expression of TTLL4 in PDAC Cells

Among dozens of trans-activated genes that were screened by genome-wide cDNA microarray analysis of PDAC cells (Nakamura T et al., 2004 Oncogene 23: 2385-400), TTLL4 was focused on for this study. TTLL4 over-expression was confirmed by RT-PCR in four of the nine microdissected-PDAC cell populations (FIG. 1A). Northern-blot analysis using an TTLL4 cDNA fragment as the probe identified an about 4-kb transcript only in the testis, but no expression was observed in any other organs including lung, heart, liver, kidney, and brain (FIG. 1B). The present inventors also examined TTLL4 expression in eight PDAC cell lines and found its expression in all of the examined PDAC cell lines (FIG. 1C).

2. Effect of TTLL4-siRNA on Growth of PDAC Cells

To investigate the biological significance of TTLL4 expression in PDAC cells, three siRNA-expression vectors specific to TTLL4 transcript (si#466, si#2692, and si#2146) were constructed and transfected into Panc-1 (left) or Mia-Paca-2 (right) cells that endogenously expressed high levels of TTLL4. Knockdown effect was observed by RT-PCR when si#466 and si#2692 were transfected to Panc-1 cells, but not si#2146 or a negative control siEGFP (FIG. 2A left). Colony-formation (FIG. 2B left) and MTT assays (FIG. 2C left) using Panc-1 revealed a drastic reduction in the number of cells transfected with si#466 and si#2692, compared with si#2146 and siEGFP for which no knockdown effect was observed. The similar results were obtained when these siRNA-expression vectors were transfected into Mia-Paca-2 cells (FIGS. 2A, B and C, right).

3. TTLL4 Over-Expression Promoted Cell Growth

To investigate for the potential of TTLL4 as an oncogene, wild-type TTLL4 or enzyme-dead TTLL4 (E906A) were over-expressed in COS7 cells (FIG. 3A) and the growth promoting effect was evaluated by their over-expression. Western blot analysis using GT335 antibody (FIG. 3B), which can specifically detect polyglutamate side chains (Wolff A et al., Eur J Cell Biol 1992, 59: 425-432), demonstrated that wild-type TTLL4 over-expression enhanced polyglutamylation, while enzyme-dead TTLL4 (E906A) did not. MTT assay (FIG. 3C) demonstrated that wild-type TTLL4 significantly promoted cell growth, but not enzyme-dead TTLL4 (E906A), comparing with the growth of mock-transfected cells. These results indicated that TTLL4 could promote cell growth through its enzymatic activity for polyglutamylation.

4. TTLL4 Polyglutamylated a None-Tubulin Protein, PELP1

Proteomics approach identified several putative substrates for polyglutamylation by TTLL family members (van Dijk J et al., J Bio Chem 2004, 283:3915-3922). First, TTLL4 was over-expressed in Hela cells and proteins were compared detecting by polyglutamylation-specific antibody: GT335 antibody (Wolff A et al., Eur J Cell Biol 1992, 59: 425-432). GT335 antibody detected the increased level of polyglutamylation in several proteins including alpha-tubulin and beta-tubulin around 60 kDa-band (FIG. 4A). Next, the change of polyglutamylation pattern was checked in PDAC cells in TTLL4 knockdown by using GT335. GT335 antibody detected several polyglutamylated proteins including alpha-tubulin and beta-tubulin around 60-kDa bands in Mia-PaCa-2 (FIG. 4B right) and KLM-1 cell lines (FIG. 4B left). When TTLL4 was knocked down in Mia-PaCa-2 and KLM-1 cell lines, only 200 kDa-band was diminished commonly in both cell lines (FIG. 4B), which was also detected in over-expression of TTLL4 (FIG. 4A arrow). Among the candidate polyglutamylated proteins identified by the proteomic approach (van Dijk J et al., 2008 J Bio Chem 283:3915-3922), this 200 kDa-band was likely to correspond to PELP1 (prolin-glutamic acid-leucine-rich protein 1). To confirm the polyglutamylation of PELP1 by TTLL4, PELP1 was immunoprecipitated from the cell lysates when TTLL4 was over-expressed (FIG. 4C) or when TTLL4 was knocked down (FIG. 4D). As shown in FIGS. 4C and 4D, western blot analysis using GT335 antibody validated PELP1 polyglutamylation level was highly concordant with TTLL4 expression, indicating that PELP1 could be polyglutamilated by TTLL4.

5. Polyglutamylation of the Glutamate-Rich Stretch Region of PELP1

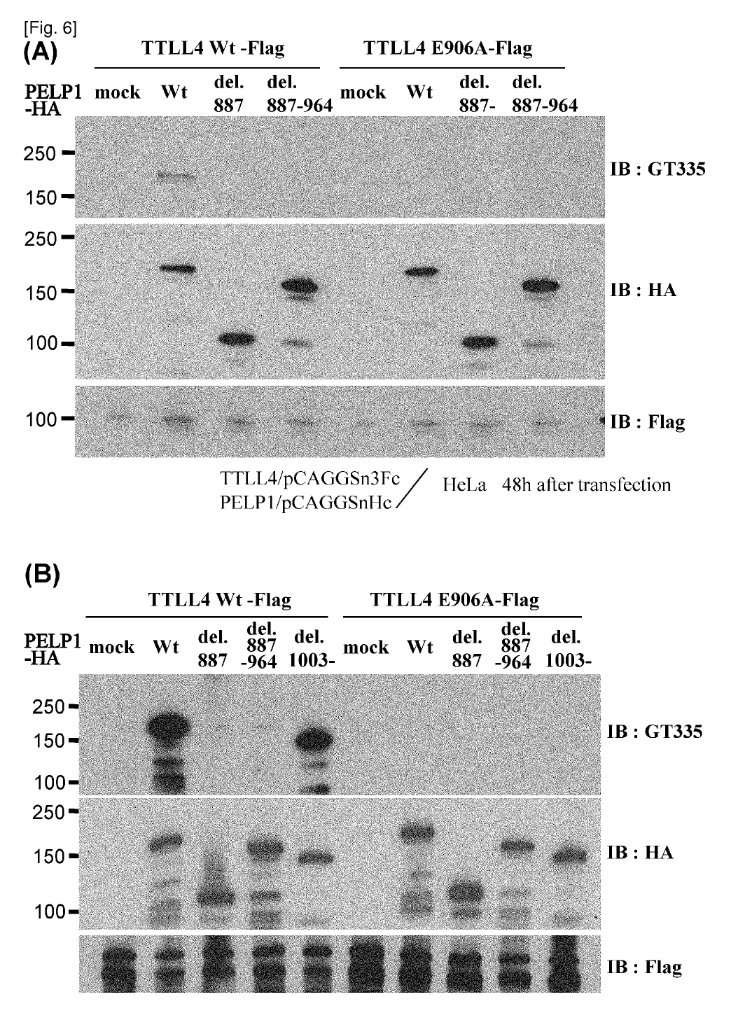

Polyglutamylation is likely to occur in the glutamate-rich stretch region of tubulins and NAPs by the side-chain manner, and PELP1 had the highly glutamate-rich region in its C-terminal region (77% in codon 887-964 is occupied by glutamate, FIG. 5A), which is a candidate of polyglutamylation region in PELP1. To confirm the polyglutamylation of this PELP1 region, three deletion constructs of PELP1 (del 887-, del 887-964, and del 1003-) were constructed, as shown in FIG. 5B. The constructs of PELP1 del 887- (lacking codon 887-) and PELIP1 del 887-964 (lacking codon 887-964) are lacking the glutamate-rich region. Each of these PELP1 constructs was co-transfected with wild-type TTLL4 expression vector or enzyme-dead TTLL4 expression vector. GT335 antibody detected the polyglutamylated PELP1 when the full-length PELP1 or the deletion construct of PELP1 del 1003- were co-transfected with wild-type TTLL4 (FIG. 6B), while other partial PELP1 lacking the glutamate-rich region or enzyme-dead TTLL4 were co-transfected, but not in the two mutant PELP1 (FIG. 6A, 6B). These findings suggested that PELP1 was likely to be polyglutamylated in its highly glutamate-rich region in its C-terminal region (codon 887-964).

6. PELP1 Complex could Interact with Histone H3 and May Involve Chromatin Re-Modeling

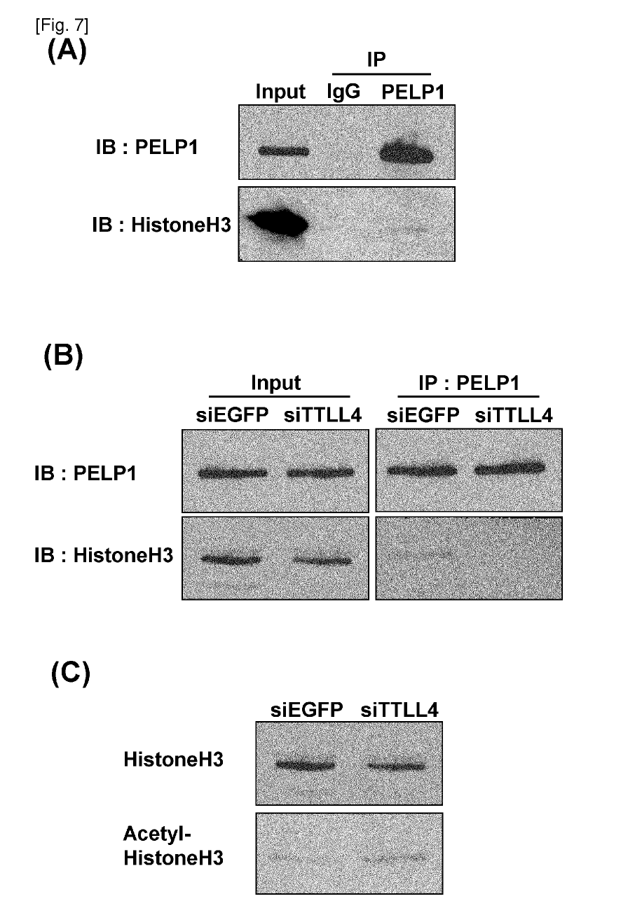

Interaction of the glutamate-rich stretch region of PELP1 and histone has been reported (Nair S S et al., 2004 Cancer Res 64: 6416-23, Choi Y B et al., 2004 J Biol Chem 279: 50930-41). It was also validated the interaction between PELP1 and histone H3 by immunoprecipitation in PDAC cells (FIG. 7A). To investigate how PELP1 polyglutamylation could involve the interaction between PELP1 and histone H3, immunoprecipitation of PELP1 from the cell lysates was performed when TTLL4 was knocked down in HeLa cell and the interaction between PELP1 and histone H3 was examined. As shown in FIG. 7B, the interaction of PELP1 with histone H3 was diminished in TTLL4 knockdown (siTTLL4), compared with the control (siEGFP). PELP1 is likely to interact with HDAC2 (histone deacetylase 2) and form large complexes regulating histone modifications and chromatin remodeling. Thus, the acetylation and methylation status of histone H3 in TTLL4 knockdown was evaluated. Again, TTLL4 knockdown resulted in decrease of PELP1 polyglutamylation, and as shown in FIG. 7C, the acetylation level of histone H3 was increased in TTLL4 knockdown (siTTLL4), compared with the control (siEGFP). The methylation status in several H3 sites was not changed in TTLL4 knockdown (data not shown).

7. PELP1 Interacted with SENP3 and LAS1L, and its Polyglutamulation could Affect this Interaction

PELP1 protein has been shown to interact with various proteins such as estrogen receptor, SRC, p85, etc., and to be involved with several signaling pathways as a scaffold protein (Vadlamudi R K, Kumar R. Nucl Recept Signal; 5: e004). To further clarify the function of PELP1 polyglutamylation in PDAC cells, it was first attempted to identify novel proteins interacting with PELP1 in cancer cells. Protein complexes were immunoprecipitaed by anti-PELP1 antibody from the lysates of PDAC cells, and LC-MS/MS analysis identified LAS1L (LAS1-like), SENP3 (SUMO/sentrin/SMT3 specific protease 3), and TEX10 (testis expressed 10) to be candidate proteins interacting with PELP1 protein in PDAC cells.

Second, to validate the interaction between PELP1 and these candidate proteins, there was transfected any one of the vectors expressing PELP1-HA, LAS1L-Flag or both vectors together into COS-7 cells, and a protein complex containing PELP1 and/or LAS1L-Flag was immunoprecipitated from the cell extracts by anti-HA antibody (FIG. 8A middle) or anti-Flag antibody (FIG. 8A lower). Western blot using anti-HA antibody indicated that PELP1-HA was co-immunoprecipitated with LAS1L-Flag when the both expression vectors were co-transfected (FIG. 8A left). Furthermore, western blot using anti-Flag antibody indicated that LAS1L-Flag was co-immunoprecipitated with PELP1-HA when the both expression vectors were co-expressed (FIG. 8A right). Interaction between PELP1 and SENP3 was also validated by the similar manner (FIG. 8B). It was confirmed that PELP1-HA protein was co-immunoprecipitaed with TEX10-Flag protein, but not vice versa (FIG. 8C).

Third, to further investigate how PELP1 polyglutamylation could affect the interaction of PELP1 with LAS1L or SENP3, PELP1-HA expression vector and LAS1L or SENP3 expression vector were co-transfected with wild-type TTLL4 or enzyme-dead TTLL4 (E906A) expression vector, and a protein complex was immunoprecipitated by anti-HA antibody (PELP1-HA). As shown in FIG. 9A upper, the interaction of PELP1 with LAS1L was enhanced in wild-type TTLL4 over-expression, comparing with that in enzyme-dead TTLL4 over-expression. Concordantly, the interaction of PELP1 with LAS1L was diminished in TTLL4 knockdown (siTTLL4), as shown in FIG. 9A lower. On the other hand, the interaction of PELP1 with SENP3 was diminished in wild-type TTLL4 over-expression, comparing with that in enzyme-dead TTLL4 over-expression (FIG. 9B upper), and knockdown of TTLL4 enhanced the interaction of PELP1 with SENP3 (FIG. 9B lower). These suggested that polyglutamylation of PELP1 could affect the interaction of PELP1 with LAS1L and SENP3 and the functions of these protein complex.

Discussion

In this invention, a novel molecular target was identified for the development of pancreatic cancer treatment. Among the normal adult organs, TTLL4 is expressed in the testis and pancreatic cancer cell as shown in FIG. 1. These issues would be critical to select a molecular target for a novel therapeutic approach with minimal side effect, and in this aspect TTLL4 is a promising molecular target for pancreatic cancer treatment.

It was demonstrated that TTLL4 and its enzyme activity play important roles in cancer cell viability and growth and that TTLL4 acts as an oncogene. Most importantly, it was shown that TTLL4 polyglutamylates an oncogenic scaffold protein PELP1 (Vadlamudi R K et al., Cancer Res 2005, 65: 7724-7732; Rajhans R et al., Cancer Res 2007, 67: 5505-512; Cheskis B J et al., Steroid 2008, 73: 901-905) and that TTLL4 functions as an oncogene through polyglutamylation of PELP1 and other proteins. PELP1 appears to interact with several key molecules of cancer cell growth, such as Src, estrogen receptor, p85 PI3K (Vadlamudi R K et al., Cancer Res 2005, 65: 7724-7732; Rajhans R et al., Cancer Res 2007, 67: 5505-512; Cheskis B J et al., Steroid 2008, 73: 901-905), and several signaling pathways appear to cross-talk in the scaffold of PELP1.

Furthermore, it was suggested that PELP1 interacts with histone H3, and polyglutamylation of PELP1 have an influence on the interaction of PELP1 and hisotne H3 and acetylation of histone H3. PELP1 and its interacting proteins, LAS1L, SENP3, and TEX10 can be included as the members of MLL1-WDR5 complex (Dou Y et al., Cell 2005, 121: 873-85; Dou Y et al., Nat Struct Mol Biol 2006, 13: 713-9), which is reported to have both histone methyltransferase activity and histone acetyltransferase activity and these activity of histone modification is likely to be highly coordinated to regulate the target transcription (Cheskis B J et al., Steroid 2008, 73: 901-905; Dou Y et al., Cell 2005, 121: 873-85). Polyglutamylation of PELP1 also could affect the affinity of PELP1 and these interacting proteins, and acetylation level of histone H3.

Without wishing to be bound by theory, it is noted that glutamate is an acidic amino acid with a negative charge. Polyglutamylation can change the charge of the target protein and thus change the protein confirmation drastically, leading to changes in protein function or protein-protein interactions.

### INDUSTRIAL APPLICABILITY

The gene-expression analysis of pancreatic cancer described herein, obtained through a genome-wide cDNA microarray, has identified specific genes as targets for cancer prevention and therapy. Based on the expression of a subset of these differentially expressed genes, the present invention provides molecular diagnostic markers for identifying and detecting cancer.

The methods described herein are also useful in the identification of additional molecular targets for prevention, diagnosis and treatment of cancer. The data reported herein add to a comprehensive understanding of cancer, facilitate development of novel diagnostic strategies, and provide clues for identification of molecular targets for therapeutic drugs and preventative agents. This invention contributes to a more profound understanding of pancreatic tumorigenesis, and provide indicators for developing novel strategies for diagnosis, treatment, and ultimately prevention of cancer.

All patents, patent applications, and publications cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method of detecting or diagnosing cancer in a subject, comprising the step of determining an expression level of the TTLL4 gene in a subject derived biological sample, wherein an increase of said level compared to a normal control level of TTLL4 indicates that said subject suffers from or is at risk of developing cancer, wherein the expression level is determined by any one method selected from the group consisting of
(a) detecting an mRNA of the TTLL4 gene;
(b) detecting a protein encoded by the TTLL4 gene; and
(c) detecting the biological activity of a protein encoded by the TTLL4 gene.

2. The method of claim 1, wherein said increase is at least 10% greater than said normal control level.

3. The method of claim 1, wherein the cancer is pancreatic cancer.

4. A kit for detecting or diagnosing cancer, which comprises a reagent selected from the group consisting of
(a) a reagent for detecting an mRNA of the TTLL4 gene;
(b) a reagent for detecting a protein encoded by the TTLL4 gene; and
(c) a reagent for detecting the biological activity of a protein encoded by the TTLL4 gene.

5. The kit of claim 4, wherein the reagent is a probe to a gene transcript of the TTLL4 gene.

6. The kit of claim 4, wherein the reagent is an antibody against the protein encoded by the TTLL4 gene.

7. The kit of claim 4, wherein the cancer is pancreatic cancer.

8-19. (canceled)

20. A method of screening for a candidate compound for treating or preventing cancer relating to TTLL4, or inhibiting TTLL4 expressing cancer cell growth, said method comprising the steps of:
(a) contacting a test compound with a polypeptide encoded by a polynucleotide of TTLL4;
(b) detecting the binding activity between the polypeptide and the test compound, or detecting the biological activity of the polypeptide of step (a); and
(c) selecting the test compound that binds to the polypeptide, or selecting the test compound that suppresses the biological activity of the polypeptide encoded by the polynucleotide of TTLL4 as compared to the biological activity of said polypeptide detected in the absence of the test compound.

21. (canceled)

22. The method of claim 20, wherein the biological activity is selected from the group consisting of facilitation of cell proliferation and polyglutamylation activity.

23. A method of screening for a candidate compound for treating or preventing cancer, relating to TTLL4 or inhibiting TTLL4 expressing cancer cell growth, said method comprising the steps of:
(a) contacting a test compound with a cell expressing TTLL4; and
(b) selecting the test compound that reduces the expression level of TTLL4 in comparison with the expression level detected in the absence of the test compound.

24. A method of screening for a candidate compound for treating or preventing cancer relating to TTLL4 or inhibiting TTLL4 expressing cancer cell growth, said method comprising the steps of:
(a) contacting a test compound with a cell into which a vector, comprising the transcriptional regulatory region of TTLL4 and a reporter gene that is expressed under the control of the transcriptional regulatory region, has been introduced;
(b) measuring the expression or activity of said reporter gene; and
(c) selecting the test compound that reduces the expression or activity level of said reporter gene as compared to the expression or activity of said reporter gene detected in the absence of the test compound.

25. A method of screening for a candidate compound for treating or preventing cancer relating to TTLL4 or inhibiting TTLL4 expressing cancer cell growth, said method comprising the steps of:
(a) contacting a TTLL4 polypeptide or functional equivalent thereof with a PELP1 polypeptide or functional equivalent thereof, in the presence of a test compound;
(b) detecting the binding between the polypeptides; and
(c) selecting the test compound that inhibits the binding between the polypeptides.

26. The method of claim 25, wherein the functional equivalent of TTLL4 polypeptide comprises a PELP1 binding domain.

27. The method of claim 25, wherein the functional equivalent of PELP1 polypeptide comprises a TTLL4 binding domain.

28. A method of screening for a candidate compound for treating or preventing cancer relating to TTLL4 or inhibiting TTLL4 expressing cancer cell growth, said method comprising the steps of:
(a) contacting a TTLL4 polypeptide or functional equivalent thereof with a substrate to be polyglutamylated and a glutamate as a cofactor in the presence of a test compound under conditions suitable for polyglutamylation of the substrate;
(b) detecting the polyglutamylation level of the substrate; and
(c) selecting the test compound that reduces the polyglutamylation level as compared to the polyglutamylation level detected in the absence of the test compound.

29. The method of claim 28, wherein the functional equivalent of TTLL4 polypeptide comprises TTL domain corresponding to SEQ ID NO: 22.

30. The method of claim 28, wherein the substrate is the polypeptide comprising glutamate rich domain corresponding to SEQ ID NO: 23.

31. The method of claim 28, wherein the substrate is PELP1.

32. A method of screening for a candidate compound for treating or preventing cancer relating to TTLL4 or inhibiting TTLL4 expressing cancer cell growth, said method comprising the steps of:
(a) contacting a PELP1 polypeptide or functional equivalent thereof with either a LAS1L or SENP3 polypeptide, or functional equivalent thereof, in the presence of a TTLL4 polypeptide or functional equivalent thereof, and a test compound;
(b) detecting the binding activity between the PELP1 polypeptide and either the LASIL or SENP3 polypeptide; and
(c) selecting the test compound that inhibits the binding activity between the PELP1 polypeptide and the LASIL polypeptide as compared to the binding activity between PELP1 polypeptide and the LASIL polypeptide in the absence of the test compound or selecting the test compound that enhances the binding activity between the PELP1 polypeptide and the SENP3 polypeptide as compared to the binding activity between PELP1 polypeptide and the SENP3 polypeptide in the absence of the test compound.

33. (canceled)

34. The method of claim 32, wherein the functional equivalent of the TTLL4 polypeptide comprises a TTL domain corresponding to SEQ ID NO: 22.

35. The method of claim 32, wherein the functional equivalent of the PELP1 polypeptide comprises a glutamate rich domain corresponding to SEQ ID NO: 23.

36. The method of claim 20, wherein the cancer is pancreatic cancer.

37. A kit for screening for a candidate compound for treating or preventing cancer relating to TTLL4 or inhibiting TTLL4 expressing cancer cell growth, wherein the compound reduces polyglutamylation activity, said kit comprising the components of:
(a) a TTLL4 polypeptide or functional equivalent thereof;
(b) a substrate capable of polyglutamylation by the polypeptide of (a);
(c) glutamate; and
(d) a reagent for detecting the polyglutamylation of substrate.

38. The kit of claim 37, wherein the functional equivalent of TTLL4 polypeptide comprises a TTL domain corresponding to SEQ ID NO: 22.

39. The kit of claim 37, wherein the substrate is the polypeptide comprising glutamate rich domain corresponding to SEQ ID NO: 23.

40. The kit of claim 37, wherein the reagent of (d) is an antipolyglutamylation antibody.

41. The method of claims 23, wherein the cancer is pancreatic cancer.

42. The method of claims 24, wherein the cancer is pancreatic cancer.

43. The method of claims 25, wherein the cancer is pancreatic cancer.

44. The method of claims 28, wherein the cancer is pancreatic cancer.

45. The method of claims 32, wherein the cancer is pancreatic cancer.
